(19) United States
(12) Patent Application Publication
(10) Pub. No.: US 2020/0015832 A1
(43) Pub. Date: Jan. 16, 2020

(54) SURGICAL SYSTEM WITH IMPROVED LARYNGEAL BLADE

(71) Applicant: MEDROBOTICS CORPORATION, Raynham, MA (US)

(72) Inventors: Lewis J. Levine, Weston, MA (US); Richard Owen Kuenzler, Framingham, MA (US); Jamie Matthew Maietta, Taunton, MA (US); Liam O'Shea, Westwood (MA); Richard L. Walker, JR., West Bridgewater, MA (US); Thomas Smith, Fall River, MA (US); Giorgio Brusa, Needham, MA (US); R. Maxwell Flaherty, Auburndale, FL (US); J. Christopher Flaherty, Auburndale, FL (US)

(21) Appl. No.: 16/497,619

(22) PCT Filed: Apr. 4, 2018
(86) PCT No.: PCT/US2018/026016

§ 371 Date: Sep. 25, 2019

Related U.S. Application Data
(60) Provisional application No. 62/481,309, filed on Apr. 4, 2017.
(60) Provisional application No. 62/598,812, filed on Dec. 14, 2017.
(60) Provisional application No. 62/617,513, filed on Jan. 15, 2018.

(51) Int. Cl. A61B 17/24; A61B 13/00

(52) U.S. Cl. CPC A61B 17/24; A61B 1/24; A61B 13/00

(57) ABSTRACT

A system for performing a medical procedure comprises: a retraction device for maintaining an opening into a patient's mouth; a tool for performing the medical procedure; and an articulating probe. The retraction device comprises a tissue manipulation element. The articulating probe delivers the tool through the retractor-maintained opening, alongside the tissue manipulation element, and to a target location of the patient. Methods and retractor devices are also provided.

### RELATED APPLICATIONS

This application claims benefit of U.S. Provisional Application No. 62/481,309, filed Apr. 4, 2017, the content of which is incorporated herein by reference in its entirety.

This application claims benefit of U.S. Provisional Application No. 62/598,812, filed Dec. 14, 2017, the content of which is incorporated herein by reference in its entirety.

This application claims benefit of U.S. Provisional Application No. 62/617,513, filed Jan. 15, 2018, the content of which is incorporated herein by reference in its entirety.

This application is related to U.S. Provisional Application No. 61/921,858, filed Dec. 30, 2013, the content of which is incorporated herein by reference in its entirety.

This application is related to PCT Application No PCT/US2014/071400, filed Dec. 19, 2014, the content of which is incorporated herein by reference in its entirety.

This application is related to U.S. patent application Ser. No. 14/892,750, filed Nov. 20, 2015, the content of which is incorporated herein by reference in its entirety.

This application is related to U.S. patent application Ser. No. 15/899,826, filed Feb. 20, 2018, the content of which is incorporated herein by reference in its entirety.

This application is related to U.S. Provisional Application No. 61/406,032, filed Oct. 22, 2010, the content of which is incorporated herein by reference in its entirety.

This application is related to PCT Application No PCT/US2011/057282, filed Oct. 21, 2011, the content of which is incorporated herein by reference in its entirety.

This application is related to U.S. patent application Ser. No. 13/880,525, filed Apr. 19, 2013, now U.S. Pat. No. 8,992,421, the content of which is incorporated herein by reference in its entirety.

This application is related to U.S. patent application Ser. No. 14/587,166, filed Dec. 31, 2014, the content of which is incorporated herein by reference in its entirety.

This application is related to U.S. Provisional Application No. 61/492,578, filed Jun. 2, 2011, the content of which is incorporated herein by reference in its entirety.

This application is related to PCT Application No. PCT/US12/40414, filed Jun. 1, 2012, the content of which is incorporated herein by reference in its entirety.

This application is related to U.S. patent application Ser. No. 14/119,316, filed Nov. 21, 2013, the content of which is incorporated herein by reference in its entirety.

This application is related to U.S. Provisional Application No. 62/504,175, filed May 10, 2017, the content of which is incorporated herein by reference in its entirety.

This application is related to U.S. Provisional Application No. 61/412,733, filed Nov. 11, 2010, the content of which is incorporated herein by reference in its entirety.

This application is related to PCT Application No PCT/US2011/060214, filed Nov. 10, 2011, the content of which is incorporated herein by reference in its entirety.

This application is related to U.S. patent application Ser. No. 13/884,407, filed May 9, 2013, now U.S. Pat. No. 9,649,163, the content of which is incorporated herein by reference in its entirety.

This application is related to U.S. patent application Ser. No. 15/587,832, filed May 5, 2017, the content of which is incorporated herein by reference in its entirety.

This application is related to U.S. Provisional Application No. 61/472,344, filed Apr. 6, 2011, the content of which is incorporated herein by reference in its entirety.

This application is related to PCT Application No. PCT/US12/32279, filed Apr. 5, 2012, the content of which is incorporated herein by reference in its entirety.

This application is related to U.S. patent application Ser. No. 14/008,775, filed Sep. 30, 2013, the content of which is incorporated herein by reference in its entirety.

This application is related to U.S. patent application Ser. No. 14/944,665, filed Nov. 18, 2015, the content of which is incorporated herein by reference in its entirety.

This application is related to U.S. patent application Ser. No. 14/945,685, filed Nov. 19, 2015, the content of which is incorporated herein by reference in its entirety.

This application is related to U.S. Provisional Application No. 61/534,032 filed Sep. 13, 2011, the content of which is incorporated herein by reference in its entirety.

This application is related to PCT Application No. PCT/US12/54802, filed Sep. 12, 2012, the content of which is incorporated herein by reference in its entirety.

This application is related to U.S. patent application Ser. No. 14/343,915, filed Mar. 10, 2014, now U.S. Pat. No. 9,757,856, issued Sep. 12, 2017, the content of which is incorporated herein by reference in its entirety.

This application is related to U.S. patent application Ser. No. 15/064,043, filed Mar. 8, 2016, now U.S. Pat. No. 9,572,628, issued Feb. 21, 2017, the content of which is incorporated herein by reference in its entirety.

This application is related to U.S. patent application Ser. No. 15/684,268, filed Aug. 23, 2017, the content of which is incorporated herein by reference in its entirety.

This application is related to U.S. Provisional Application No. 61/368,257, filed Jul. 28, 2010, the content of which is incorporated herein by reference in its entirety.

This application is related to PCT Application No PCT/US2011/044811, filed Jul. 21, 2011, the content of which is incorporated herein by reference in its entirety.

This application is related to U.S. patent application Ser. No. 13/812,324, filed Jan. 25, 2013, now U.S. Pat. No. 9,901,410, issued Feb. 27, 2018, the content of which is incorporated herein by reference in its entirety.

This application is related to U.S. patent application Ser. No. 15/874,189, filed Jan. 18, 2018, the content of which is incorporated herein by reference in its entirety.

This application is related to U.S. Provisional Application No. 61/578,582, filed Dec. 21, 2011, the content of which is incorporated herein by reference in its entirety.

This application is related to PCT Application No. PCT/US12/70924, filed Dec. 20, 2012, the content of which is incorporated herein by reference in its entirety.

This application is related to U.S. patent application Ser. No. 14/364,195, filed Jun. 10, 2014, now U.S. Pat. No. 9,364,955 issued Jun. 14, 2016, the content of which is incorporated herein by reference in its entirety.

This application is related to U.S. patent application Ser. No. 15/180,503, filed Jun. 13, 2016, now U.S. Pat. No. 9,821,477, issued Nov. 21, 2017, the content of which is incorporated herein by reference in its entirety.

This application is related to U.S. patent application Ser. No. 15/786,901, filed Oct. 18, 2017, the content of which is incorporated herein by reference in its entirety.

This application is related to U.S. Provisional Application No. 61/681,340, filed Aug. 9, 2012, the content of which is incorporated herein by reference in its entirety.

This application is related to PCT Application No. PCT/US13/54326, filed Aug. 9, 2013, the content of which is incorporated herein by reference in its entirety.

This application is related to U.S. patent application Ser. No. 14/418,993, filed Feb. 2, 2015, now U.S. Pat. No. 9,675,380 issued Jun. 13, 2017, the content of which is incorporated herein by reference in its entirety.

This application is related to U.S. patent application Ser. No. 15/619,875, filed Jun. 12, 2017, the content of which is incorporated herein by reference in its entirety.

This application is related to U.S. Provisional Application No. 61/751,498, filed Jan. 11, 2013, the content of which is incorporated herein by reference in its entirety.

This application is related to PCT Application No. PCT/US14/10808, filed Jan. 9, 2014, the content of which is incorporated herein by reference in its entirety.

This application is related to U.S. patent application Ser. No. 14/759,020, filed Jan. 9, 2014, the content of which is incorporated herein by reference in its entirety.

This application is related to U.S. Provisional Application No. 61/656,600, filed Jun. 7, 2012, the content of which is incorporated herein by reference in its entirety.

This application is related to PCT Application No. PCT/US13/43858, filed Jun. 3, 2013, the content of which is incorporated herein by reference in its entirety.

This application is related to U.S. patent application Ser. No. 14/402,224, filed Nov. 19, 2014, the content of which is incorporated herein by reference in its entirety.

This application is related to U.S. Provisional Application No. 61/825,297, filed May 20, 2013, the content of which is incorporated herein by reference in its entirety.

This application is related to PCT Application No. PCT/US13/38701, filed May 20, 2014, the content of which is incorporated herein by reference in its entirety.

This application is related to U.S. patent application Ser. No. 14/888,541, filed Nov. 2, 2015, now U.S. Pat. No. 9,517,059, issued Dec. 13, 2016, the content of which is incorporated herein by reference in its entirety.

This application is related to U.S. patent application Ser. No. 15/350,549, filed Nov. 14, 2016, the content of which is incorporated herein by reference in its entirety.

This application is related to U.S. Provisional Application No. 61/818,878, filed May 2, 2013, the content of which is incorporated herein by reference in its entirety.

This application is related to PCT Application No. PCT/US14/36571, filed May 2, 2014, the content of which is incorporated herein by reference in its entirety.

This application is related to U.S. patent application Ser. No. 14/888,189, filed Oct. 30, 2015, now U.S. Pat. No. 9,913,695, issued Mar. 13, 2018, the content of which is incorporated herein by reference in its entirety.

This application is related to U.S. patent application Ser. No. 14/916,664, filed Mar. 9, 2018, the content of which is incorporated herein by reference in its entirety.

This application is related to U.S. Provisional Application No. 61/909,605, filed Nov. 27, 2013, the content of which is incorporated herein by reference in its entirety.

This application is related to U.S. Provisional Application No. 62/052,736, filed Sep. 19, 2014, the content of which is incorporated herein by reference in its entirety.

This application is related to PCT Application No. PCT/US14/67091, filed Nov. 24, 2014, the content of which is incorporated herein by reference in its entirety.

This application is related to U.S. patent application Ser. No. 15/038,531, filed May 23, 2016, the content of which is incorporated herein by reference in its entirety.

This application is related to U.S. Provisional Application No. 62/008,453 filed Jun. 5, 2014, the content of which is incorporated herein by reference in its entirety.

This application is related to PCT Application No. PCT/US15/34424, filed Jun. 5, 2015, the content of which is incorporated herein by reference in its entirety.

This application is related to U.S. patent application Ser. No. 15/315,868, filed Dec. 2, 2016, the content of which is incorporated herein by reference in its entirety.

This application is related to U.S. Provisional Application No. 62/150,223, filed Apr. 20, 2015, the content of which is incorporated herein by reference in its entirety.

This application is related to U.S. Provisional Application No. 62/299,249, filed Feb. 24, 2016, the content of which is incorporated herein by reference in its entirety.

This application is related to PCT Application No. PCT/US16/28374, filed Apr. 20, 2016, the content of which is incorporated herein by reference in its entirety.

This application is related to U.S. patent application Ser. No. 15/567,109, filed Oct. 17, 2017, the content of which is incorporated herein by reference in its entirety.

This application is related to U.S. Provisional Application No. 62/401,390, filed Sep. 29, 2016, the content of which is incorporated herein by reference in its entirety.

This application is related to PCT Application No. PCT/US17/54297, filed Sep. 29, 2017, the content of which is incorporated herein by reference in its entirety.

This application is related to U.S. Provisional Application No. 62/517,433, filed Jun. 9, 2017, the content of which is incorporated herein by reference in its entirety.

This application is related to U.S. Provisional Application No. 62/533,644, filed Jul. 17, 2017, the content of which is incorporated herein by reference in its entirety.

This application is related to U.S. Provisional Application No. 62/614,263, filed Jan. 5, 2018, the content of which is incorporated herein by reference in its entirety.

This application is related to U.S. Provisional Application No. 62/582,283, filed Nov. 6, 2017, the content of which is incorporated herein by reference in its entirety.

This application is related to U.S. Provisional Application No. 62/614,346, filed Jan. 5, 2018, the content of which is incorporated herein by reference in its entirety.

This application is related to U.S. Provisional Application No. 62/613,899, filed Jan. 5, 2018, the content of which is incorporated herein by reference in its entirety.

This application is related to U.S. Provisional Application No. 62/614,223, filed Jan. 5, 2018, the content of which is incorporated herein by reference in its entirety.

This application is related to U.S. Design application No. 29/632,148, filed Jan. 5, 2018, the content of which is incorporated herein by reference in its entirety.

This application is related to U.S. Provisional Application No. 62/614,224, filed Jan. 5, 2018, the content of which is incorporated herein by reference in its entirety.

This application is related to U.S. Provisional Application No. 62/614,228, filed Jan. 5, 2018, the content of which is incorporated herein by reference in its entirety.

This application is related to U.S. Provisional Application No. 62/614,225, filed Jan. 5, 2018, the content of which is incorporated herein by reference in its entirety.

This application is related to U.S. Provisional Application No. 62/614,240, filed Jan. 5, 2018, the content of which is incorporated herein by reference in its entirety.

This application is related to U.S. Provisional Application No. 62/614,235, filed Jan. 5, 2018, the content of which is incorporated herein by reference in its entirety.

This application is related to U.S. patent application Ser. No. 11/630,279, filed Dec. 20, 2006, published as U.S. Patent Application Publication No. 2009/0171151, the content of which is incorporated herein by reference in its entirety.

### SUMMARY

Embodiments of the systems, devices and methods described herein can be directed to systems, devices and methods for performing a medical procedure through a natural orifice or skin incision. A retractor includes a tongue depressor blade constructed and arranged to minimize forces applied to tissue and to provide access for an articulating probe.

According to an aspect of the present inventive concepts, a retraction device for maintaining an opening into a patient's mouth comprises a tissue manipulation element. The tissue manipulation element comprises a proximal portion with a first axis and a distal portion with a second axis. The first axis and the second axis define an interior angel, and the interior angle comprises an angle less than 120°.

According to another aspect of the present inventive concepts, a system for performing a medical procedure comprises a retraction device for maintaining an opening into a patient's mouth, comprising a tissue manipulation element; a tool for performing the medical procedure; and an articulating probe for delivering the tool through the retractor maintained opening, alongside the tissue manipulation element, and to a target location of the patient. The tissue manipulation element comprises a proximal portion with a first axis and a distal portion with a second axis. The first axis and the second axis define an interior angel, and the interior angle comprises an angle less than 120°.

According to another aspect of the present inventive concepts, a system for performing a medical procedure comprises a human interface device for receiving commands from an operator; a controller operably attached to the human interface device; and a retraction device comprising a tissue manipulation element operatively attached to the controller. Manipulation of the human interface device by the operation causes the controller to robotically position the tissue manipulation element.

In some embodiments, the tissue manipulation element is constructed and arranged to apply a force to at least tissue of the tongue and/or epiglottis.

In some embodiments, the interior angle comprises an angle less than or equal to 110°, or less than or equal to 100°, or less than or equal to 900.

In some embodiments, the tissue manipulation element is constructed and arranged to minimize forces applied to tissue while providing a sufficient opening to allow passage of an introducer device. The tissue manipulation element can be constructed and arranged to minimize forces applied to the tongue and/or epiglottis. The introducer device can comprise the articulating probe.

In some embodiments, the tissue manipulation element comprises a geometry that approximates a portion of the patient's anatomy such that forces applied to the anatomy are distributed along a majority of the length of the tissue manipulation element to avoid areas of highly concentrated force. The portion of the patient's anatomy approximated can include tissue proximate the tongue, epiglottis, and/or vallecula.

In some embodiments, the tissue manipulation element comprises a material selected from the group consisting of: stainless steel; aluminum; titanium; plastic; and combinations thereof.

In some embodiments, the tissue manipulation element comprises an element for delivering and/or removing a fluid. The tissue manipulation element can comprise an element for removing smoke.

In some embodiments, the tissue manipulation element comprises a proximal portion and a distal portion. The proximal portion and the distal portion can provide a relatively continuous curve. The continuous curve has a radius of curvature between 1″ and 5″, or between 2″ and 4″. The proximal portion and the distal portion can have different radii of curvature. The proximal portion has a radius of curvature between 1″ and 3″, or approximately 2.2″. The distal portion has a radius of curvature between 2″ and 4″, or approximately 3.1″. The tissue manipulation element can further comprise a connecting portion proximal to and connected to the proximal portion. The connecting portion can comprise a radius of curvature between 5″ and 10″, or approximately 7.4″. The proximal portion and the distal portion can collectively comprise a path length between 100 mm and 140 mm, or approximately 124 mm. The distal portion can comprise a tip portion at its distal end. The proximal portion can comprise a proximal axis, and the tip portion can be located an orthogonal distance of between 1″ and 4″ from the proximal axis, approximately 2.66″ from the proximal axis. The tip portion can be located an orthogonal distance of at least 1″ from the proximal axis, or at least 2″ from the proximal axis, or at least 3″ from the proximal axis. The proximal portion and distal portion can collectively comprise a tapered geometry. The proximal portion can comprise a proximal end with a width between 1″ and 3″.

In some embodiments, the tissue manipulation element comprises a top surface and a bottom surface, and the top surface includes a convex profile along its minor axis, and the bottom surface comprises a concave profile along its minor axis.

In some embodiments, the tissue manipulation element comprises a length between 4.5″ and 8″, or approximately 6.1″.

In some embodiments, the tissue manipulation element comprises an adjustable geometry. The tissue manipulation element can comprise a robotically adjustable geometry.

In some embodiments, the tissue manipulation element comprises at least one functional element. The functional element can comprise a sensor and/or a transducer. The functional element can comprise a sensor selected from the group consisting of: a pressure sensor; a smoke sensor; a pH sensor; a blood gas sensor; blood glucose sensor; a respiration sensor; an EEG sensor; a temperature sensor; an electrode; and combinations thereof. The functional element can comprise a sensor configured to provide tissue manipulation element shape information. The functional element can comprise a transducer selected from the group consisting of: a light; an infrared light; a visible light; a radioactive element; an ultrasound delivery element; an electrode; a camera; and combinations thereof. The functional element can comprise a light and/or a camera configured to provide illumination and/or visualization of neighboring anatomy.

In some embodiments, the tissue manipulation element comprises two or more segments connected by one or more hinges, and rotation of the one or more hinges adjusts the shape of the tissue manipulation element. The tissue manipulation element shape can be adjusted to a continuous curve, a discontinuous curve, and/or a complex shape. The system and/or retraction device can further comprise at least one motor attached to at least one of the one or more hinges and constructed and arranged to rotate one or more of the segments to adjust the shape of the tissue manipulation element. The system and/or retraction device can further comprise a controller and a human interface device collectively configured to robotically adjust the shape of the tissue manipulation element. The at least one motor can comprise a gear reduction assembly. The system and/or retraction device can further comprise at least one cable attached to at least one segment and constructed and arranged to rotate the at least one segment about at least one hinge. The system and/or retraction device can further comprise at least one motor constructed and arranged to advance, retract, and/or adjust the tension of the at least one cable. The at least one motor can comprise a gear reduction assembly. The system and/or retraction device can further comprise a controller and a human interface device collectively configured to robotically adjust the shape of the tissue manipulation element.

In some embodiments, the retractor device is constructed and arranged provide non-line-of-sight access to the target location, the articulating probe is constructed and arranged to translate through the non-line-of-sight-access, and the tool is constructed and arranged to travel through a working channel of the articulating probe, and perform the medical procedure at the target location. The working channel can comprise an external working channel and/or an internal working channel.

In some embodiments, the retraction device comprises an oral retraction device.

In some embodiments, the retraction device comprises an articulation assembly constructed and arranged to articulate the tissue manipulation element with at least three degrees of freedom. The retraction device can comprise a support element constructed and arranged to provide a stabilizing force to the articulation assembly during articulation of the tissue manipulation element.

In some embodiments, the retraction device comprises an articulation assembly constructed and arranged to rotationally articulate the tissue manipulation element. The retraction device can comprise a support element constructed and arranged to provide a stabilizing force to the articulation assembly during articulation of the tissue manipulation element.

In some embodiments, the target location comprises a location selected from the group consisting of: the nasal passage; throat; oropharynx; larynx; esophagus; vocal chord; trachea; stomach; and combinations thereof.

In some embodiments, the system and/or retraction device further comprises an introducer assembly comprising the articulating probe and a feeder unit, the feeder unit constructed and arranged to advance, retract, and/or steer the articulating probe. The introducer assembly can comprise a robotic introducer assembly. The introducer assembly can comprise a controller and a human interface device. The introducer assembly can be constructed and arranged to change the position of the tissue manipulation element. The introducer assembly can be constructed and arranged to modify the shape of the tissue manipulation element. The human interface device can comprise a user input component selected from the group consisting of: a multi-axis input device such as a joystick; a mouse; a keyboard; a touchscreen; a haptic input device; a manipulatable arm; and combinations thereof. The human interface device can comprise a user output component selected from the group consisting of: a display; a touchscreen; a light; an audible alert element; a haptic feedback element such as a vibrational, acoustic, and/or visual element; and combinations thereof.

In some embodiments, the system and/or retraction device further comprises a tool for performing a medical procedure. The tool can comprise a tool selected from the group consisting of: a grasper; a claw; scissors; a cutter; a knife; an ablator; a cauterizer; a drug delivery apparatus; a radiation source; a laser emitter; an energy delivery element such as a RF electrode; a sensor such as a pressure sensor or a blood sensor; a camera; a magnet; a heating element; a light source, a cryogenic element; and combinations thereof.

According to another aspect of the present inventive concepts, a patient access device for providing access through a patient's mouth to the throat of the patient comprises a tissue manipulation element comprising a length and a jaw manipulation element that is positionable along the length of the tissue manipulation element.

In some embodiments, the jaw manipulation element comprises a length between 30 mm and 65 mm.

In some embodiments, the tissue manipulation element comprises a length between 125 mm and 255 mm.

In some embodiments, the tissue manipulation element comprises a straight segment and a curved segment.

According to another aspect of the present inventive concepts, a patient access device for providing access through a patient's mouth to the throat of the patient comprises a tissue manipulation element comprising a proximal portion and two or more interchangeable distal portions. The first interchangeable distal portion comprises a first geometry and the second interchangeable distal portion comprises a second geometry. The first geometry is different than the second geometry.

According to another aspect of the present inventive concepts, a patient access device for providing access through a patient's mouth to the throat of the patient comprises a tissue manipulation element, an articulation assembly constructed and arranged to articulate the tissue manipulation element with at least three degrees of freedom, and a support element constructed and arranged to provide a stabilizing force to the articulation assembly during the articulation of the tissue manipulation element. The patient access device is constructed and arranged such that the patient is in a gallows suspension position when supported by the tissue manipulation element.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 illustrates an anatomic view of a system for performing a medical procedure, consistent with the present inventive concepts.

FIG. 2 illustrates an isometric view of a tongue depressor blade, consistent with the present inventive concepts.

FIGS. 2A-2C illustrate side, end and top views, respectively, of a tongue depressor blade, consistent with the present inventive concepts.

FIG. 3 illustrates an end view of a tongue depressor blade, consistent with the present inventive concepts.

FIG. 4 illustrates an isometric view of an oral retractor device, consistent with the present inventive concepts.

FIG. 5 illustrates a close-up cutaway side view of an articulation assembly of an oral retractor in a first position, consistent with the present inventive concepts.

FIG. 5A illustrates the articulation assembly of FIG. 5 in a second position, consistent with the present inventive concepts.

FIG. 5B illustrates a close-up view of a positioning assembly of FIGS. 5 and 5A illustrating a rotational adjustment of a tongue depressor blade, consistent with the present inventive concepts.

FIGS. 6A-6C illustrate graphic demonstrations of a highly articulated probe device, consistent with the present inventive concepts.

FIGS. 7A-7D illustrate side views of a series of continuous curve shape adjustments to a robotically controlled tongue depressor blade including motor-driven hinges, consistent with the present inventive concepts.

FIG. 7E illustrates a side view of a discontinuous shape adjustment of a robotically controlled tongue depressor blade, consistent with the present inventive concepts.

FIGS. 8A, 8B, 8C illustrate side views of a series of shape adjustments to a robotically controlled tongue depressor blade including cable driven segments, consistent with the present inventive concepts.

FIGS. 9A and 9B illustrate a side view of a tongue depressor blade and a tooth guard, and a side view of the tooth guard attached to the tongue depressor blade, respectively, consistent with the present inventive concepts.

FIG. 10 illustrates a side view of a tooth guard positioned on a proximal portion of a tongue depressor blade, consistent with the present inventive concepts.

FIG. 11 illustrates a side view of a tooth guard positioned on a distal portion of a tongue depressor blade, consistent with the present inventive concepts.

FIG. 12 illustrates side views of a retraction device and tongue depressor blades, and a top view of an attachment mechanism, consistent with the present inventive concepts.

FIG. 12A illustrates a perspective view of a retraction device, consistent with the present inventive concepts.

FIG. 13 illustrates a tongue depressor blade having a removable distal portion, consistent with the present inventive concepts.

FIGS. 14A and B illustrate a side view and an end view of a tongue depressor blade, respectively, consistent with the present inventive concepts.

FIGS. 15A, 15B, 15C illustrate side, top, and end views of a tongue depressor blade, respectively, consistent with the present inventive concepts.

### DETAILED DESCRIPTION OF THE DRAWINGS

Reference will now be made in detail to the present embodiments of the technology, examples of which are illustrated in the accompanying drawings. The same reference numbers are used throughout the drawings to refer to the same or like parts.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate” shall include locations relatively close to, on, in and/or within a referenced component or other location.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as light (e.g. a transducer comprising a light emitting diode or light bulb), sound (e.g. a transducer comprising a piezo crystal configured to deliver ultrasound energy), pressure, heat energy, cryogenic energy, chemical energy; mechanical energy (e.g. a transducer comprising a motor or a solenoid), magnetic energy, and/or a different electrical signal (e.g. a Bluetooth or other wireless communication element). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy (e.g. a transducer comprising one or more electrodes); light energy (e.g. a transducer comprising a laser, light emitting diode and/or optical component such as a lens or prism); mechanical energy (e.g. a transducer comprising a tissue manipulation element); sound energy (e.g. a transducer comprising a piezo crystal); chemical energy; electromagnetic energy; magnetic energy; and combinations of one or more of these.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

Provided herein are systems, devices and methods for safely and effectively accessing one or more internal body locations at which a medical procedure is performed. Retractors (e.g. oral retraction devices) of the present inventive concept can maintain an opening into the patient's mouth. The retractors can include a tongue depressor blade that comprises a proximal portion with a first axis and a distal portion with a second axis. The first axis and the second axis define an interior angle (as defined herein), such as an interior angle less than 120°. The retractors of the present inventive concepts can avoid applying excessive force to patient tissue while allowing access of an articulating probe (e.g. a robotically translatable and steerable probe comprising a set of inner links and outer links) to locations beyond the mouth (e.g. the pharynx, esophagus and/or stomach). In some embodiments, a system includes a retractor of the present inventive concepts as well as the articulating probe. In some embodiments, a system includes a human interface device for receiving commands from an operator; a controller operably attached to the human interface device; and a robotically manipulatable tongue depressor blade operatively attached to the controller. Manipulation of the human interface device by the operator causes the controller to robotically manipulate the tongue depressor blade.

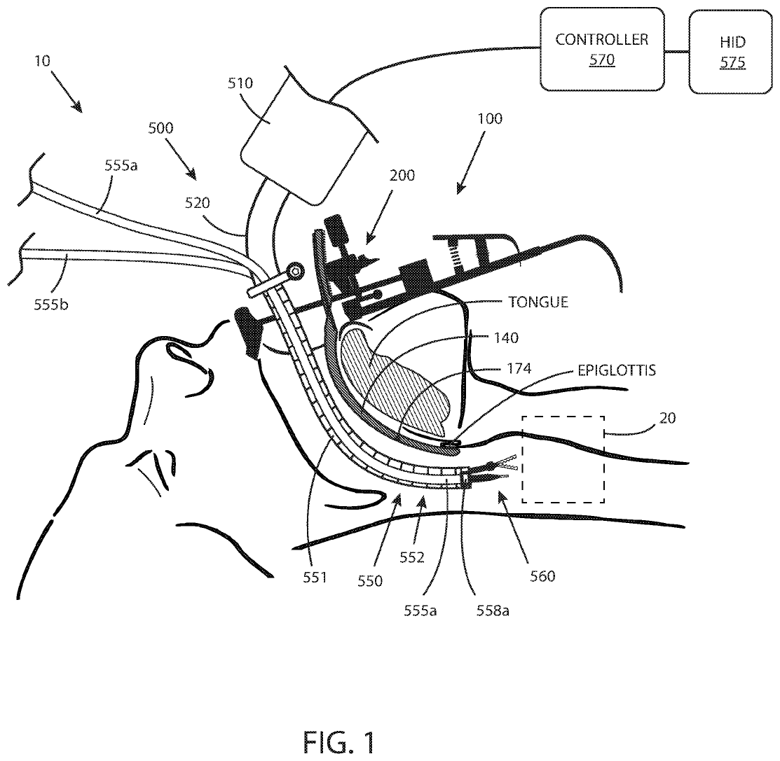

Referring to FIG. 1, an anatomical view of a system for performing a medical procedure is illustrated, consistent with the present inventive concepts. System 10 can comprise a retraction device comprising an oral retractor device 100, including a curved tissue manipulation element, tongue depressor blade 140, and an introducer assembly 500. System 10 can comprise a patient access device comprising a curved tissue manipulation element, tongue depressor blade 140, and a jaw manipulation element, tooth guard 150. In some embodiments, system 10 provides access (e.g. surgical access) to a site of a medical procedure, target location 20 shown. Target location 20 can comprise a portion of the patient's mouth and/or throat. In some embodiments, target location 20 is outside of any line-of-sight visualizable by an operator (e.g. a surgeon or other clinician) looking into the patient's mouth. Oral retractor device 100 can maintain an opening at and/or into a patient's mouth, and tongue depressor blade 140 provides access (e.g. creates an unobstructed non-line-of-sight path for subsequently introduced flexible and/or articulatable instruments, such as by displacing at least a portion of the patient's tongue and/or capturing a portion of the epiglottis) to target location 20 during a medical procedure. In some embodiments, system 10 comprises: an oral retractor device 100 comprising a tongue depressor 140, the oral retractor device 100 providing non-line-of-sight access (e.g. non-linear access) to a target location 20; an articulating probe 550 constructed and arranged to translate through the non-line-of-sight access provided by the oral retractor device 100 (e.g. travel in a curvilinear path); and a tool 560 (e.g. a flexible tool) constructed and arranged to travel through a working channel of the articulating probe 550 to the target location 20 and perform a medical procedure.

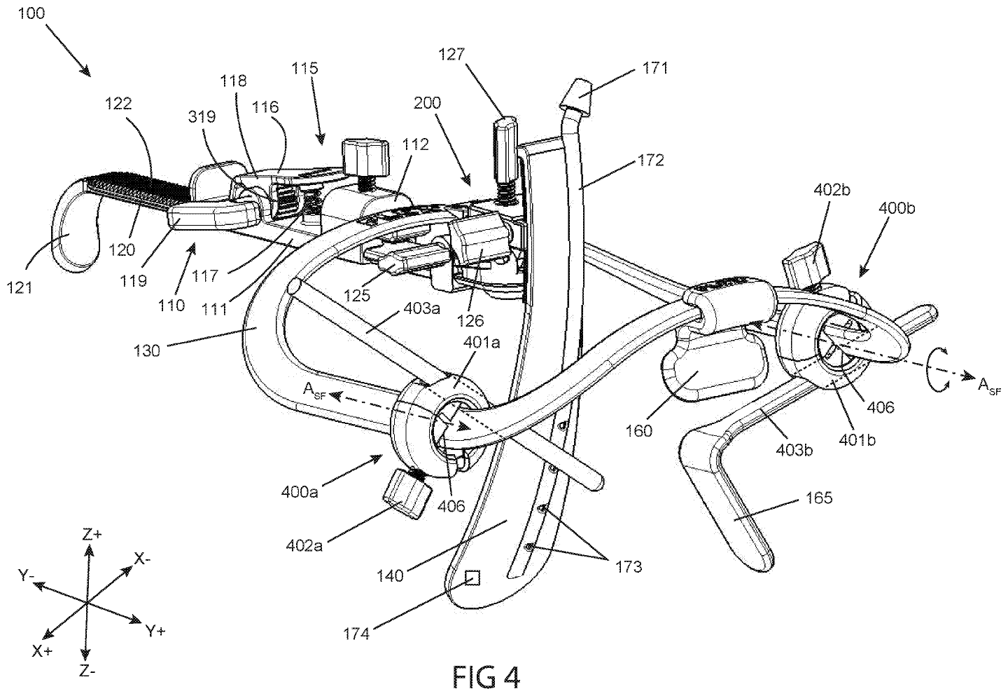

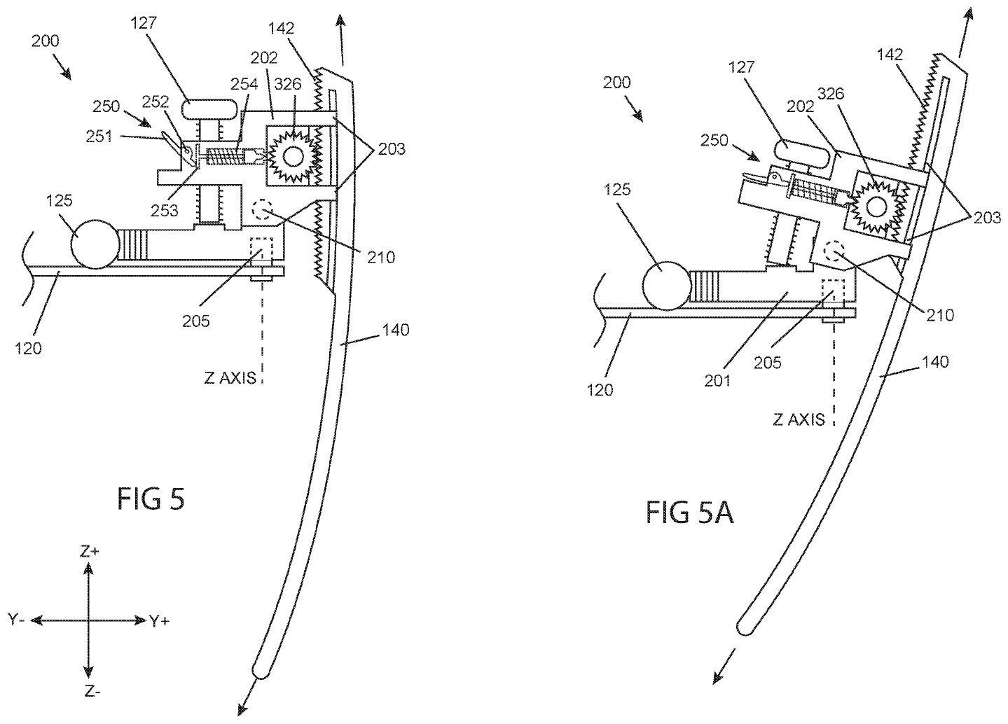

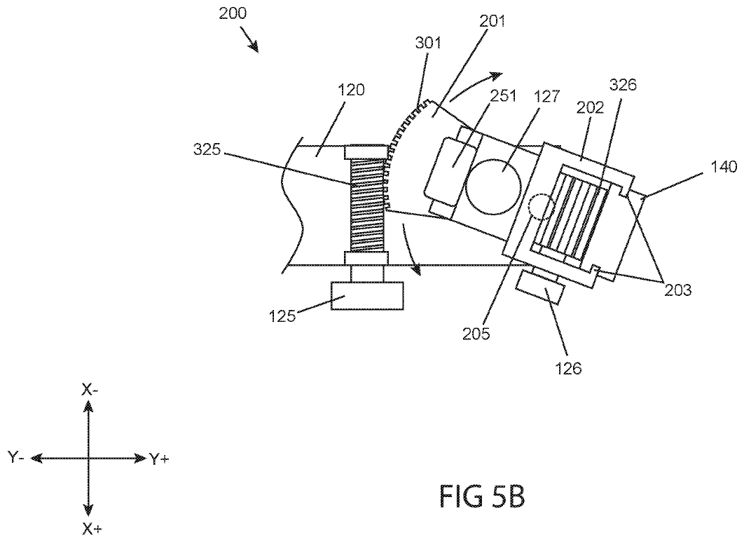

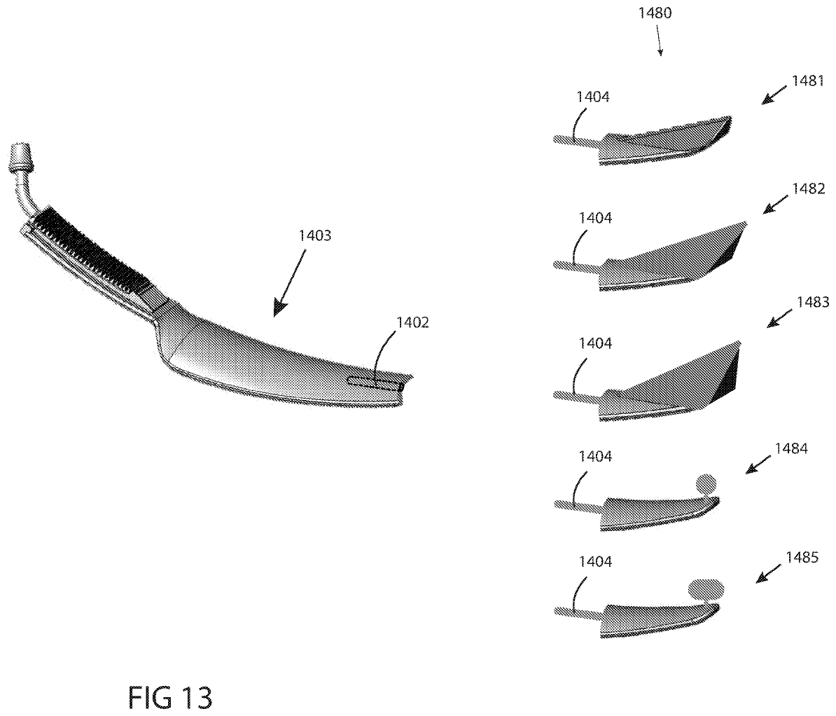

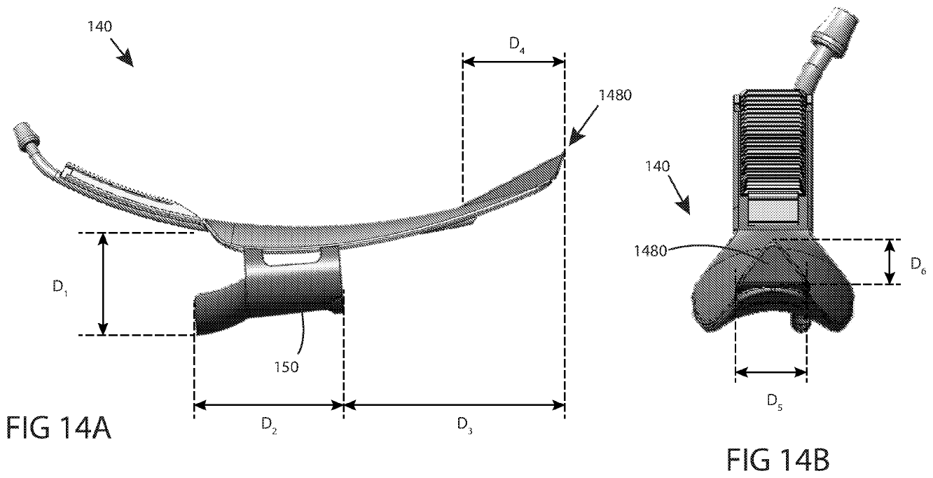

Tongue depressor blade 140 can be constructed and arranged to manipulate (e.g. move such as to create an opening and/or capture to prevent from blocking an opening) tissue of the tongue, the epiglottis, and/or any other tissue of the body to which blade 140 is in contact. In some embodiments, oral retractor device 100 comprises an articulation assembly 200 constructed and arranged to manipulate (e.g. rotate) the position of tongue depressor blade 140 relative to the patient, such as is described herebelow in reference to FIGS. 4, 5A and/or 5B. Oral retractor device 100 can be used to provide access for an introducer device, introducer assembly 500 shown, and/or related surgical tools, to target location 20. Introducer assembly 500 can be constructed and arranged to follow the curved contour of tongue depressor blade 140 to access target location 20. Tongue depressor blade 140 and/or other portions of oral retractor device 100 can be constructed and arranged to place the patient in a modified suspension state that has reduced forces applied to tissue of the patient as compared to conventional laryngoscope devices and/or suspension methods (e.g. a gallows suspension). Tongue depressor blade 140 can be constructed and arranged such that its distal tip is placed at and/or distal to the epiglottis and/or vallecula (e.g. to selectively capture tissue proximate the epiglottis and/or vallecula). Tongue depressor blade 140 can include a geometry (e.g. contour) that closely approximates a portion of the patient's anatomy (e.g. a portion of the anatomy proximate the tongue, epiglottis, and/or vallecula), thereby distributing forces along a majority of tongue depressor blade 140 length, and thus avoiding areas of highly concentrated force (e.g. avoiding applying a concentrated force to the epiglottis). In some embodiments, tongue depressor blade 140 is constructed and arranged to minimize forces applied to tissue while providing a sufficient opening to allow passage of an introducer device, such as articulating probe 550. In some embodiments, tongue depressor blade 140 comprises a tip geometry constructed and arranged to capture the epiglottis (e.g. between blade 140 and base of the patient's tongue, as shown), while minimizing force applied to the epiglottis. Examples of tip geometries are described herebelow in reference to FIGS. 13 and 14.

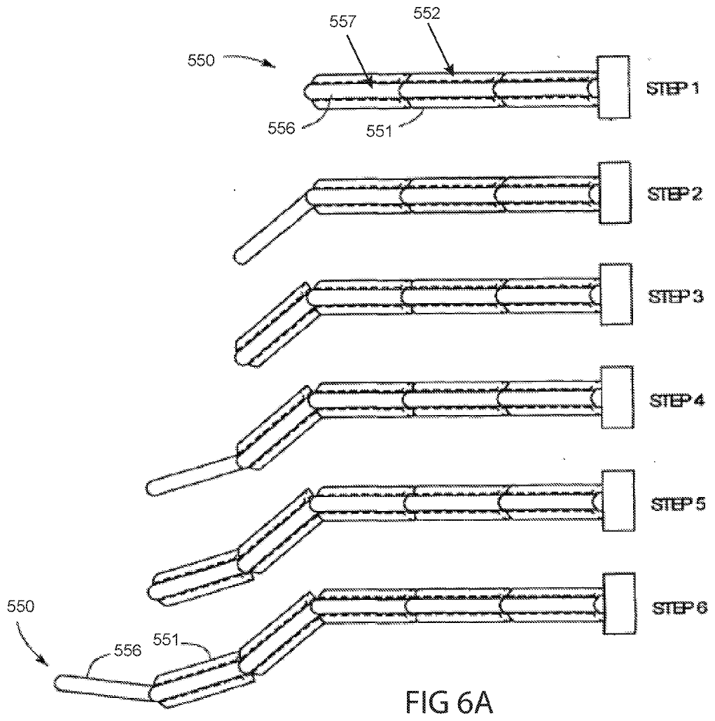

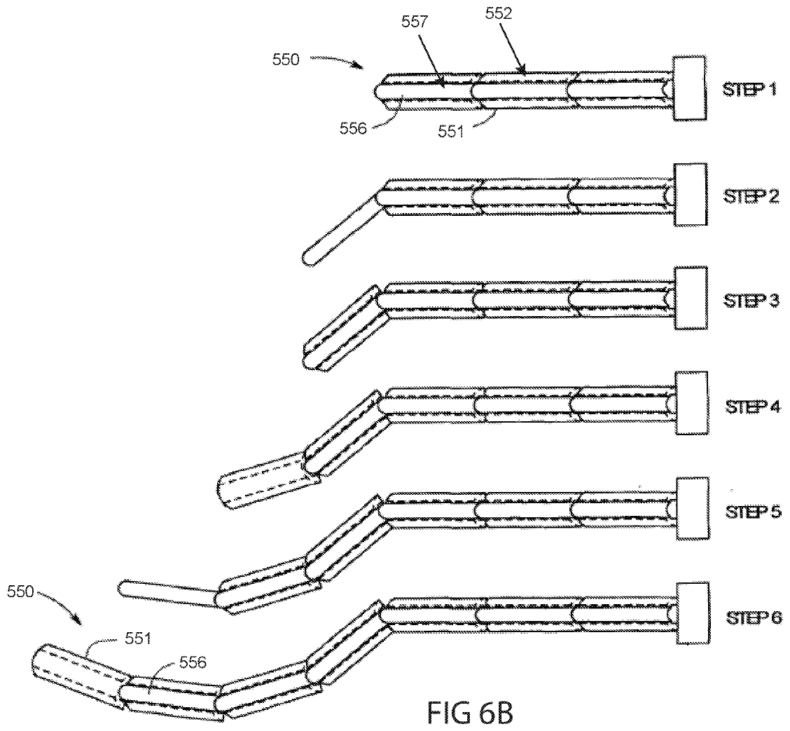

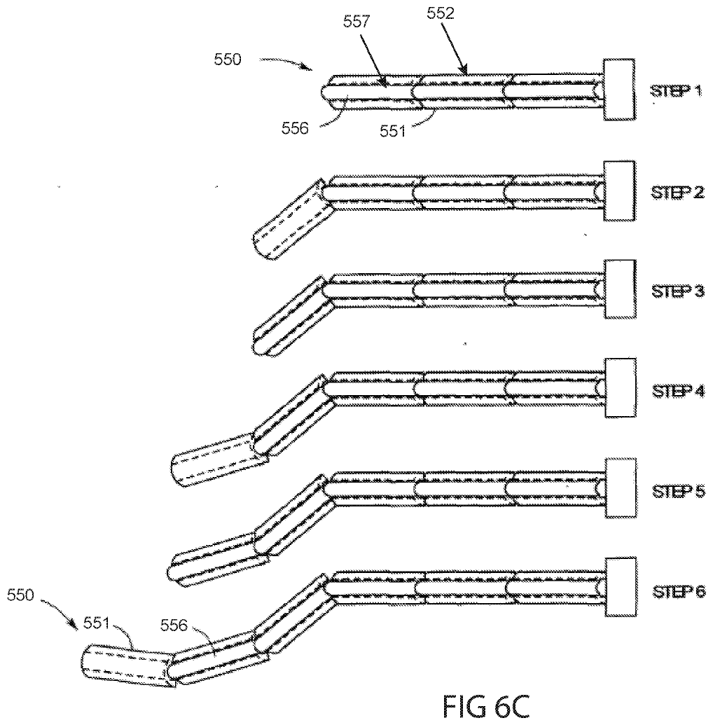

Introducer assembly 500 can comprise a robotic introducer assembly. In some embodiments, introducer assembly 500 comprises a feeder unit 510, an introducer portion 520, and an articulating probe 550. Introducer portion 520 can be constructed and arranged to slidingly receive articulating probe 550, and subsequently support, stabilize, and/or otherwise guide articulating probe 550 toward target location 20. For example, feeder unit 510 and introducer portion 520 can be positioned such that articulating probe 550 exits introducer 520 into the opening of the patient's mouth. In some embodiments, the distal portion of introducer 520 is positioned within the patient's mouth, orienting articulating probe 550 towards target location 20 (and/or a circuitous pathway toward target location 20). Articulating probe 550 can comprise an assembly of rotating links that includes an outer link mechanism 552 comprising a plurality of outer links 551, and an inner link mechanism 557 comprising a plurality of inner links 556, such as those described herebelow in reference to FIGS. 6A-C, and/or as described in applicant's co-pending U.S. patent application Ser. No. 14/587,166, titled “Highly Articulated Robotic Probes and Methods of Production and Use of Such Probes”, filed Dec. 31, 2014 (Attorney Docket Number MED-026-US-CON1), the content of which is incorporated herein by reference in its entirety for all purposes. Articulating probe 550 can be constructed and arranged to exit the distal end of introducer portion 520, drive or otherwise extend along a user defined articulated path (as described herein), reach target location 20, and establish a surgical platform beyond the reach of a line-of-sight surgical platform. Introducer assembly 500 can further include a controlling assembly, controller 570 shown, which can include a human interface device, HID 575. Controller 570 can comprise electronic circuitry, mechanical linkages and other mechanisms, and/or other controlling assemblies that manipulate one or more portions of feeder unit 510 (e.g. manipulate motors, pulleys, screws, drives, cables and/or other components of feeder unit 510). HID 575 can comprise one or more user input components, such as a user input component selected from the group consisting of: a multi-axis input device such as a joystick; a mouse; a keyboard; a touchscreen; a haptic input device; a manipulatable arm; and combinations of one or more of these. HID 575 can comprise one or more user output components, such as a user output component selected from the group consisting of: a display; a touchscreen; a light; an audible alert element; a haptic feedback element such as a vibrational, acoustic, and/or visual element; and combinations of one or more of these. An operator, such as a surgeon, interfaces with HID 575 which advances, retracts, steers, and/or otherwise manipulates probe 550 via feeder unit 510, such as is described in applicant's co-pending U.S. patent application Ser. No. 13/812,324, titled “Surgical Positioning and Support System”, filed Jan. 9, 2014 (Attorney Docket Number MED-049-US), the content of which is incorporated herein by reference in its entirety for all purposes.

In some embodiments, system 10 further includes one or more tools 560 (e.g. the two tools shown in FIG. 1). In some embodiments, a tool 560 can be inserted through feeder unit 510, into an internal working channel (not shown but positioned within articulating probe 550, such as between an inner link mechanism and outer link mechanism of probe 550 as described herein). In these embodiments, each tool 560 exits the distal end of probe 550, such as to access target location 20. Alternatively or additionally, introducer 500 can include one or more guide tubes 555 (e.g. tubes 555a and 555b shown), which include a lumen which functions as a working channel external to probe 550. The distal portion of each tube 555 is attached (e.g. fixedly, slidingly and/or rotatably attached) to a channel within one, two or more radial projections from probe 550, side ports 558 (e.g. side ports 558a shown, and 558b not shown but approximately 180° circumferentially offset from 558a). Side ports 558 can be fixedly attached to or integral to (e.g. in a monolithic arrangement) at least the distal portion of probe 550. Tool 560 can be inserted into the proximal end of an external working channel of a guide tube 555, and exit its distal end, at a location proximate the surgical platform provided by system 10 at target location 20. Tool 560 can comprise one, two or more tools selected from the group consisting of: a grasper; a claw; scissors; a cutter; a knife; an ablator; a cauterizer; a drug delivery apparatus; a radiation source; a laser emitter; an energy delivery element such as a RF electrode; a sensor such as a pressure sensor or a blood sensor; a camera; a magnet; a heating element; a light source, a cryogenic element; and combinations of these.

In some embodiments, tongue depressor blade 140 comprises a curved blade, such as a blade with a proximal portion with a first axis and a distal portion with a second axis that is angularly offset from the first axis (e.g. at an interior angle. as defined herein, of less than 120°), such as is described herebelow. In some embodiments, tongue depressor blade 140 comprises a material selected from the group consisting of: stainless steel; aluminum; titanium; plastic; and combinations of one or more of these.

In some embodiments, tongue depressor blade 140 comprises a tube or lumen for delivering and/or removing a fluid (e.g. smoke), such as is described herebelow in reference to tube 172, port 171 and holes 173 of FIG. 4.

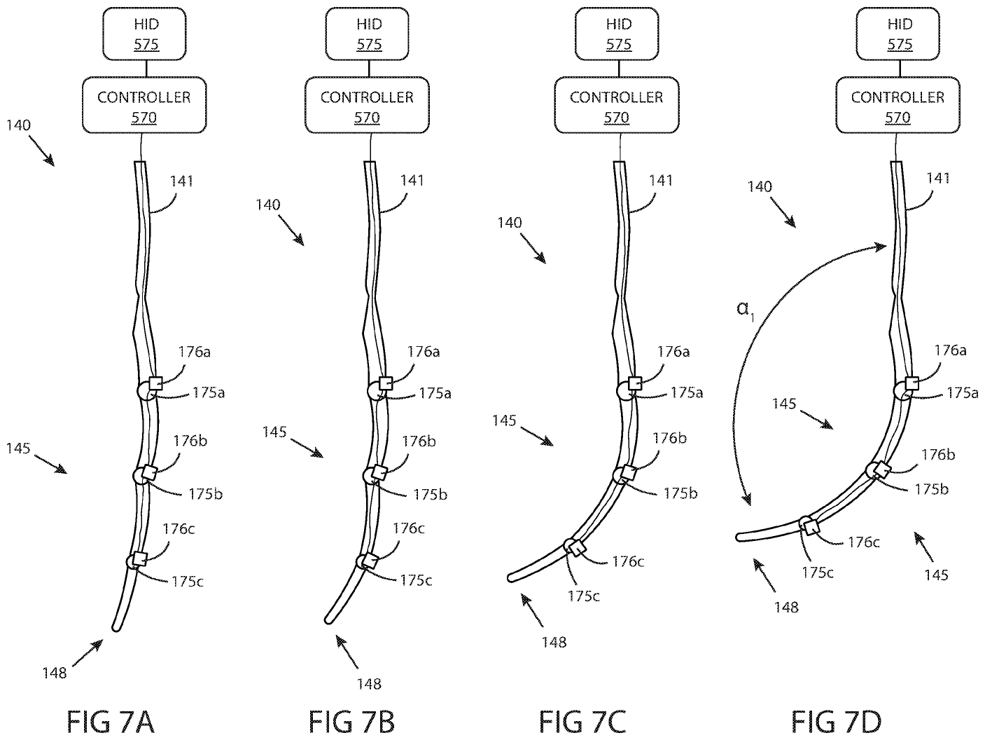

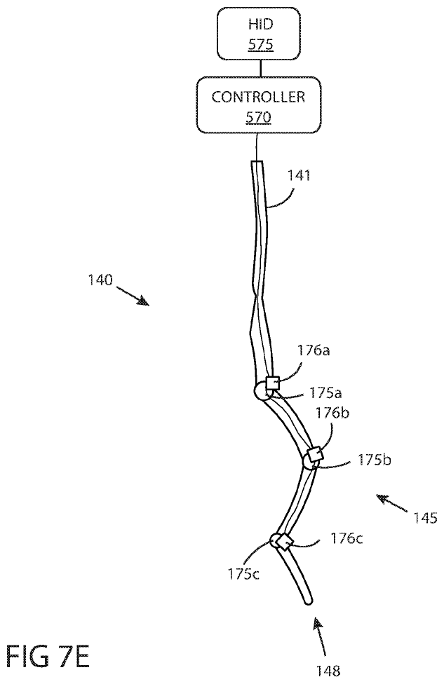

In some embodiments, tongue depressor blade 140 comprises a functional element, functional element 174 shown. Functional element 174 can comprise one or more sensors or transducers (e.g. as described hereabove) positioned on or near a surface of tongue depressor 140. Alternatively or additionally, one or more functional elements 174 can be positioned on or near fluid attachment port 171 and/or tube 172. Functional element 174 can comprise a sensor selected from the group consisting of: a pressure sensor; a smoke sensor; a pH sensor; a blood gas sensor; blood glucose sensor; a respiration sensor; an EEG sensor; a temperature sensor; an electrode; and combinations of these. Alternatively or additionally, functional element 174 can comprise a transducer selected from the group consisting of: a light; an infrared light; a visible light; a radioactive element; an ultrasound delivery element; an electrode; a camera; and combinations of these. Functional element 174 can be attached to an electronic control assembly of system 10, such as controller 570, such as to transmit or receive signals to or from controller 570, such as to provide one or more functions in a closed-loop manner. In some embodiments, tongue depressor blade 140 is constructed and arranged to have its shape operatively adjusted (e.g. as described herebelow in reference to FIGS. 7A-D, 7E and/or 8A-C), and functional element 174 comprises a sensor configured to provide shape information (e.g. to provide closed-loop or other control of blade 140 shape). In some embodiments, functional element 174 comprises a light delivery element (e.g. a light emitting diode) and/or a camera, such as to provide illumination and/or to provide visualization of neighboring anatomy (e.g. at least a portion of target location 20 or tissue proximate to target location 20) to an operator (e.g. to aid in placement of tongue depressor blade 140 or manipulation of one or more tools 560).

In some embodiments, tongue depressor blade 140 comprises a non-conductive material (e.g. an electrically non-conductive material), or includes at least a non-conductive portion (e.g. an electrically non-conductive portion).

In some embodiments, tongue depressor blade 140 comprises an adjustable geometry, such as a robotically adjustable shape as described herebelow in reference to FIGS. 7A-D, 7E and/or 8A-C.

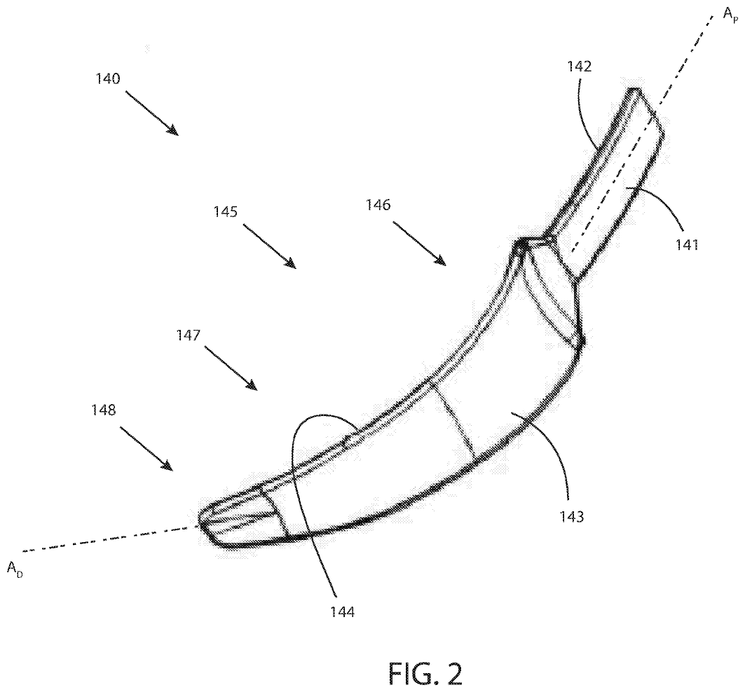

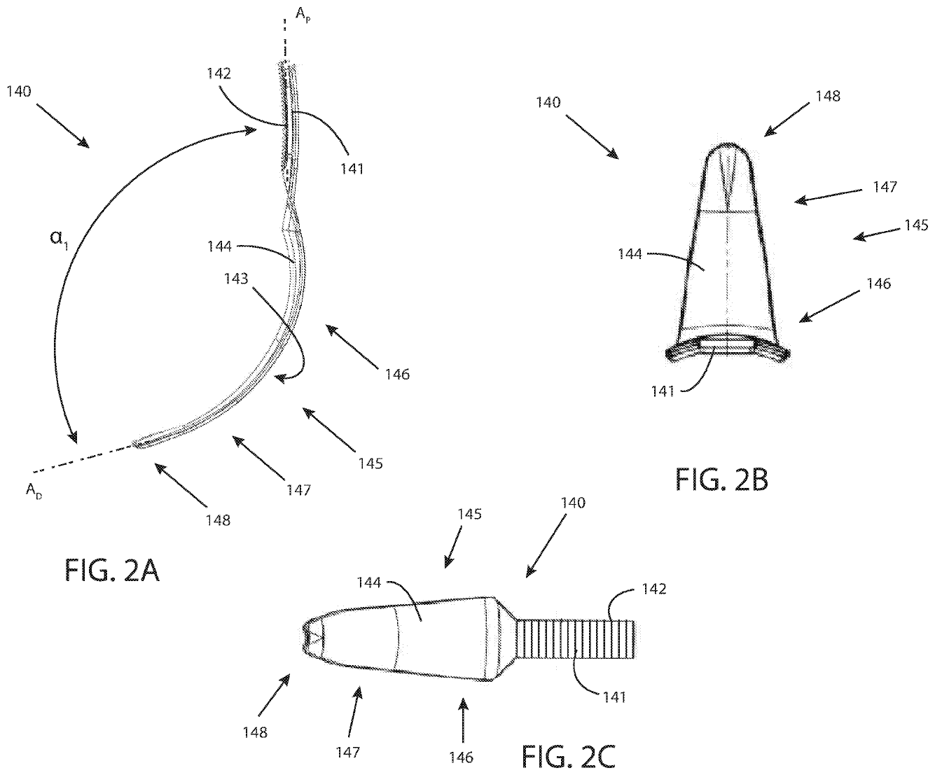

Referring now to FIGS. 2 and 2A-C, various views of a curved tongue depressor blade are illustrated, consistent with the present inventive concepts. In FIG. 2, an isometric view of tongue depressor blade 140 is illustrated, consistent with the present inventive concepts. In FIGS. 2A-C, a side, an end, and a top view of tongue depressor blade 140 are illustrated, respectively, also consistent with the present inventive concepts. Tongue depressor blade 140 comprises a connecting portion 141, which operably connects blade 140 to an oral retractor, such as a connection to oral retractor 100 via articulation assembly 200 as described hereabove in reference to FIG. 1. Connecting portion 141 can comprise one or more attachment features, such as geared teeth 142, as shown. Tongue depressor blade 140 further comprises a blade portion 145, constructed and arranged to operably engage (e.g. capture or otherwise manipulate) at least a portion of the patient's mouth and/or throat (e.g. at least a portion of the tongue and/or at least a portion of the epiglottis).

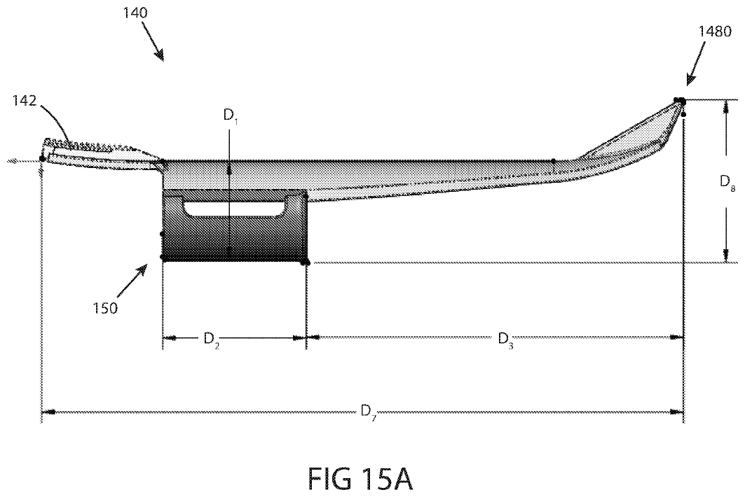

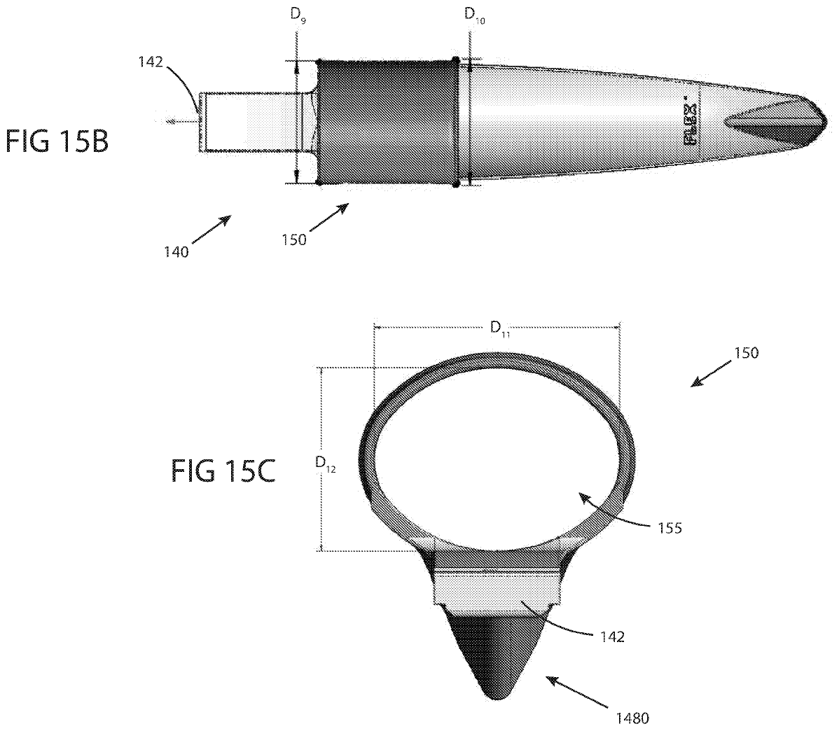

Blade portion 145 can comprise a first portion, proximal blade portion 146, and a second portion, distal blade portion 147. Distal blade portion 147 comprises a distal tip portion, tip 148. Tip 148 can comprise a geometry constructed and arranged to capture and/or manipulate at least a portion of the patient's tongue and/or epiglottis. Blade 140 further comprises a top surface 144, and a bottom surface 143. As shown, connecting portion 141 is primarily aligned with a first, proximal axis, Axis A_{P}, and distal portion 147 terminates (at tip 148) tangentially to a second, distal axis, Axis A_{D}. In some embodiments, Axis A_{D} is angularly offset from Axis A_{P}, such that an “interior angle”, the angle between Axis A_{D} and A_{P} on the inside of the curve of blade 140, angle α₁ shown, is less than or equal to 120° (e.g. a corresponding exterior angle equal to 360° minus the interior angle, is at least 240°), such as an angle α₁ that is less than or equal to 110°, 100°, and/or 90° (e.g. a corresponding exterior angle is at least 250°, 260° and/or 270° respectively). In some embodiments, angle α₁ comprises and angle less than 180°. In some embodiments, blade 140 can comprise a compound curve, such as a curve comprising at least two curved segments, each with a unique radius of curvature. In some embodiments, a first radius of curvature is approximately 250 mm, and a second radius of curvature is approximately 65 mm, and/or approximately 25% of the first radius of curvature. In some embodiments, at least a segment of blade 140 is straight, for example when a proximal segment of blade 140 is straight, and a distal segment is curved, as shown in FIG. 15A. In some embodiments, blade 140 comprises an arc length (e.g. the length along the length of a curved blade 140), the arc length comprising a length of between 30 mm and 50 mm, such as 40 mm.

In some embodiments, distal portion 147 and proximal portion 146 are arranged in a curved profile, with a curvature constructed and arranged to smoothly transition between first axis A_{P} and second axis A_{D}. In some embodiments, distal portion 147 and proximal portion 146 form a curve comprising a single, continuous curve, such as a curve with a radius of curvature between 1″ and 5″, such as between 2″ and 4″. Alternatively, proximal portion 146 can comprise a first radius of curvature, such as a radius of curvature between 1″ and 3″, such as a radius of approximately 2.2″, and distal portion 147 can comprise a second radius of curvature, such as a radius of curvature between 2″ and 4″, such as radius of approximately 3.1″. In some embodiments, connecting portion 141 comprises a radius of curvature of between 5″ and 10″, such as a radius of approximately 7.4″. In some embodiments, connecting portion 141 comprises a relatively straight geometry. In some embodiments, proximal portion 146 and distal portion 147 collectively comprise a “path length” (the length of the path along a relatively central axis of an elongate structure comprising straight and/or curved segments) of between 100 mm and 140 mm, such as a path length of approximately 124 mm. In some embodiments, connecting portion 141 comprises a path length (e.g. a relatively linear length as shown) of between 30 mm and 70 mm, such as a path length of approximately 54 mm. In some embodiments, tip 148 is positioned between 1″ and 4″ from axis A_{P} (i.e. the orthogonal distance between tip 148 and axis A_{P} is 1″ to 4″), or approximately 2.66″. In some embodiments, tip 148 is positioned (orthogonally) at least 1″, at least 2″ and/or at least 3″ from axis A_{P}.

Tongue depressor blade 140 can also comprise a curved profile along its minor axis, such that top surface 144 comprises a convex profile along its minor axis, and bottom surface 143 comprises a concave profile along its minor axis, such that blade 140 conforms to (e.g. approximates) a portion of the anatomy of the patient (e.g. more anatomic conformity than a blade with a relatively flat profile along its minor axis). Blade 140 can comprise an overall length of between 4.5″ and 8″, such as a length of approximately 6.1″. Blade 140 can comprise a tapered geometry along proximal portion 146 and distal portion 147, such that tip 148 is more narrow than the proximal end of proximal portion 146. In some embodiments, the proximal end of proximal portion 146 comprises a width of between 1″ and 3″, such as a width of approximately 1.5″. In some embodiments, connector portion 141 comprises a width of approximately 0.7″.

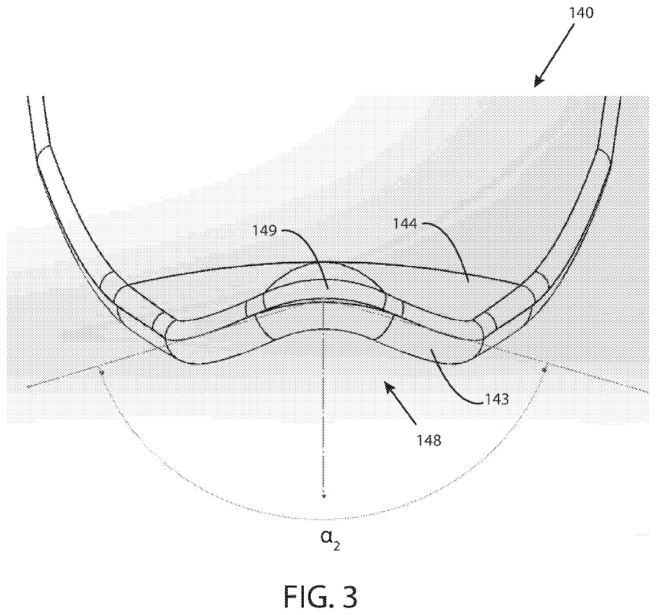

Referring additionally to FIG. 3, an end view of a tongue depressor blade is illustrated, consistent with the present inventive concepts. Tongue depressor blade 140 comprises tip 148. Tip 148 can comprise a geometry constructed and arranged to capture and/or manipulate at least a portion of the patient's epiglottis and/or vallecula. Tip 148 can comprise a triangular portion, projection 149, constructed and arranged to approximate the shape of the epiglottis and/or the vallecula of the patient. Projection 149 comprises an interior angle, angle α₂ shown, such as an angle of approximately 145°.

Referring now to FIG. 4, an isometric view of an oral retractor device is illustrated, according to the present inventive concepts. Oral retractor device 100 is positioned along X, Y, and Z axes, also referred to as frontal, longitudinal, and sagittal axes, respectively. As used herein, the X+ axis refers to an axis that can extend towards a patient's left side, the X-axis refers to an axis that can extend towards the patient's right side, the Y+ axis refers to an axis that can extend toward the patient's head, the Y− axis refers to an axis that can extend toward the patient's feet, the Z+ axis refers to an axis that can extend above the patient, and the Z− axis refers to an axis that can extend below the patient.

Oral retractor device 100 can be constructed and arranged to provide access (i.e. through the mouth) to a nasal passage, throat or related oral cavity, oropharynx, larynx, esophagus, vocal chords, trachea, and/or stomach, and/or to directly or indirectly access regions of the body proximal to the nasal passage, throat, oropharynx, larynx, esophagus, vocal chords, trachea, and/or stomach. In some embodiments, target location 20 comprises one or more of: the nasal passage, throat, oropharynx, larynx, esophagus, vocal chords, trachea, and/or stomach. Oral retractor device 100 exposes areas of a human anatomy by allowing an operator, for example, a head and neck surgeon or other medical professional, to apply forces via retractor 100 to the lips, tongue, teeth, and/or cheeks to control the geometry of the patient's mouth, for example, a size of the mouth opening. Retractor 100 is preferably in a stabilized state when applying the forces. A feature of retractor 100, in accordance with embodiments of the present inventive concepts, is that an operator (e.g., a surgeon), can gain simple and/or rapid access and exposure (via the patient's mouth) to the oropharynx, larynx, hypopharynx, and/or other internal regions of a body due to the ease of insertion and significant operator adjustability of multiple retraction elements of oral retractor device 100. Articulation of one or more positionable components of oral retractor device 100 can provide better visualization and/or access to deeper or otherwise hard-to-reach anatomical locations than would be available without the enhanced articulation.

In some embodiments, oral retractor device 100 includes an articulation assembly 200 and a support element including a support arm 120. Retractor 100 can include a linear positioning assembly 110 and a main support frame 130. Main support frame 130 can be attached to positioning assembly 110, and positioning assembly 110 can be operably attached to support arm 120, such that operation of positioning assembly 110 causes the linear translation of main support frame 130 relative to support arm 120. Main support frame 130 can comprise a closed (as shown) or open perimeter frame, constructed of one or more rigid materials, such as a metal such as stainless steel or titanium, or a rigid plastic. In some embodiments, main support frame 130 comprises a thickness of approximately 0.125″ and comprises a width of approximately 0.5″. Oral retractor device 100 can further include a tongue depressor blade 140 that is operably attached to support arm 120. Tongue depressor blade 140 can be of similar geometry or other similar construction and arrangement to any one or more tongue depressor blades 140 described herein. Oral retractor device 100 can include at least one cheek retractor 165.

Support arm 120 comprises a first support element, attachment portion 121, which can be constructed and arranged to provide a stabilizing force to oral retractor device 100. Main support frame 130 can be constructed and arranged as a second support element of oral retractor device 100, such as to provide a supporting force proximate both ends of oral retractor device 100. As described herein, tongue depressor blade 140, cheek retractors 165, and/or one or more other components of oral retractor device 100 can be disposable (e.g. disposed of after a single and/or limited number of medical procedures). In some embodiments, one or more tongue depressor blades 140 and/or other component of oral retractor device 100 can comprise a non-conductive material, such as a non-conductive plastic or metal with a non-conductive coating.

The linear positioning assembly 110 can comprise a base 111, a ratchet assembly 115, and one or more gears 319. Main support frame 130 can be coupled to an attachment point 112 at a distal end of base 111 of linear positioning assembly 110. Main support frame 130 can be formed of stainless steel, plastic, and/or other well-known material that provides structural rigidity for positioning a patient's head and mouth and maintaining the mouth in an open position. Components for retracting portions of the mouth region, such as cheek retractor 165, can be coupled to main support frame 130. Other components, such as one or more tooth engaging members, or jaw support 160, a mouth guard, and the like, can be coupled to main support frame 130. Jaw support 160 can constructed and arranged to make contact and frictionally engage with one or more of the patient's teeth (e.g. one or more of the patient's front teeth), such as to stabilize oral retractor device 100 relative to the patient. Jaw support 160, or a tooth engaging member, can be fixed and/or attachable to main support frame 130. In some embodiments, jaw support 160 comprises a set screw, not shown but constructed and arranged to selectively fix jaw support 160 to main support frame 130 (e.g. to allow translation and/or rotation of jaw support 160 relative to main support frame 130). In some embodiments, jaw support 160 comprises a rigid portion and a more flexible portion, such as a flexible portion (e.g. a soft plastic or gauze) that makes contact with the patient's teeth and/or gums.

Linear positioning assembly 110 is constructed and arranged to allow operator translation of main support frame 130 along the Y axis (e.g. to change the distance between jaw support 160 and tongue depressor blade 140). Support arm 120 comprises multiple engageable ridges, teeth 122 on its top surface as shown. Positioning assembly 110 includes gear 319 which operatively engages teeth 122. Gear 319 is attached to linear positioning knob 119 such that rotation of linear positioning knob 119 causes rotation of gear 319 about its axis and subsequent linear translation of positioning assembly 110 with respect to support arm 120. In some embodiments, positioning assembly 110 is constructed and arranged to limit translation in one or more directions. For example, positioning assembly 110 can include ratchet assembly 115 comprising lever 116 with locking portion 118, and spring assembly 117. Ratchet assembly 115 is biased by spring assembly 117 such that locking portion 118 is engaged with teeth 122. Lever 116 and locking portion 118 are constructed and arranged to allow translation of positioning assembly 110 along the Y+ axis (e.g. in a ratcheting fashion), while preventing motion along the Y− axis. When a force is applied to the proximal end of lever 116, for example, by an operator applying pressure along the Z− axis, locking portion 118 is released, or disengaged from teeth 122, permitting positioning assembly 110 to freely move along both directions of the Y axis. In some embodiments, positioning assembly 110 is constructed and arranged to translate by manually applying opposing forces to positioning assembly 110 and support arm 120, without manual rotation of linear positioning knob 119 (i.e. sufficient force to cause rotation of gear 319 along teeth 122).

Support arm 120 can be attached to tongue depressor blade 140 via articulation assembly 200. Tongue depressor blade 140 can be removably coupled to articulation assembly 200. Tongue depressor blade 140 can be disposable (e.g. single use), and/or it can be constructed and arranged to be used in two or more medical procedures. The other elements of oral retractor device 100, including at least linear positioning assembly 110, support arm 120, main support frame 130, and/or articulation assembly 200 can be constructed and arranged to be used in one, two or more medical procedures. In some embodiments, linear positioning assembly 110, support arm 120, main support frame 130, and/or articulation assembly 200 are used in more medical procedures than each tongue depressor blade 140.

Articulation assembly 200 can include a multi-axis gear assembly controlled by a rotational positioning knob 125, a height positioning knob 126, and an angular positioning knob 127 for articulating tongue depressor blade 140 in accordance with one or more degrees of freedom. Tongue depressor blade 140 can be pivoted about the X axis using angular positioning knob 127, the insertion length of tongue depressor blade 140 can be adjusted using height positioning knob 126, and/or tongue depressor blade 140 can be rotated about the Z-axis using rotational positioning knob 125. A rotation of rotational positioning knob 125 drives an articulation of tongue depressor blade 140 in a curvilinear direction with a single degree of freedom about the Z axis.

A rotation of height positioning knob 126 drives an articulation of tongue depressor blade 140 relative to articulation assembly 200 in a linear direction with a single degree of freedom along the Z axis. Accordingly, tongue depressor blade 140 can be inserted in a patient's mouth, whereby a user can move tongue depressor blade 140 up and down in the mouth to determine a linear position of blade 140 prior to coupling main support frame 130 to articulation assembly 200. A rotation of angular positioning knob 127 drives an articulation of tongue depressor blade 140 in a curvilinear direction, or pitch, with a single degree of freedom about the X axis. Accordingly, tongue depressor blade 140 can be inserted in a patient's mouth, whereby a user can move tongue depressor blade 140 up and down in the mouth to determine a desirable angular position or pitch of blade 140 prior to coupling main support frame 130 to articulation assembly 200. Engagement of a combination of one or more of rotational positioning knob 125, height positioning knob 126, and angular positioning knob 127 can permit articulation of tongue depressor blade 140 to occur with one, two, and/or three degrees of freedom and therefore enable enhanced patient access through the mouth during a medical procedure (e.g. without significant neck extension and/or without the need for a strong reclination of the patient's head). Additional modification of exposure geometry (e.g. additional degrees of freedom) can be achieved through rotation of linear positioning knob 119 which causes translation of main support frame 130 including jaw support 160 and cheek retractors 165, described herebelow.

For example, during an operation including oral retractor device 100, tongue depressor blade 140 is coupled to articulation assembly 200, and introduced into the oral cavity of the patient. The operator can move tongue depressor blade 140 up and down along the Z axis relative to articulation assembly 200 until a desired linear position is determined by rotating the height positioning knob 126. The operator can lock the linear position of blade 140 in place, then determine a desired pitch of tongue depressor blade 140 by rotating angular positioning knob 127. After the desired linear and angular position of tongue depressor blade 140 are determined, and tongue depressor blade 140 is locked in place relative to assembly 200, tongue depressor blade 140 can be removed from the oral cavity and coupled to main support frame 130. Main support frame 130 can be positioned on the patient's face, and about the patient's oral cavity for stabilizing tongue depressor blade 140. Tongue depressor blade 140 can be reinserted into the oral cavity at the previously determined height and angular position. The operator can rotate tongue depressor blade 140 axially (e.g. about the Z axis, by rotating the rotational positioning knob 125).

In some embodiments, oral retractor device 100 includes one or more tool holders, such as tool holders 400a and 400b shown attached to main support frame 130. Tool holders 400a and/or 400b can be slidingly attached (e.g. pre-attached or operator attachable and/or removable) to main support frame 130. Additionally or alternatively, tool holder 400 can be laterally attached to main support frame 130. Tool holders 400a and 400b (singly or collectively tool holder 400) each comprise a slot, slot 406 shown, which surrounds an elongate portion of main support frame 130. Tool holders 400a and 400b each comprise a passageway 401a and 401b, respectively (singly or collectively passageway 401), which can be constructed and arranged to slidingly receive a shaft portion (e.g. shaft portions 403a or 403b described herebelow) of a cheek retractor 165, a tool guide (e.g. a hollow tube constructed and arranged to slidingly receive the shaft of a tool) and/or other medical device comprising a shaft portion. Passageway 401 and/or shaft portion 403 comprises a tool guide and can be constructed and arranged to receive one or more tools selected from the group consisting of: grasper; a claw; scissors; a cutter; a knife; an ablator; a cauterizer; a drug delivery apparatus; a radiation source; a laser emitter; an energy delivery element such as a RF electrode; a sensor such as a pressure sensor or a blood sensor; a camera; a magnet; a heating element; a cryogenic element; a retractor; a retractor blade such as cheek retractor 165; and combinations of these. In some embodiments, passageway 401 comprises a diameter up to approximately 5 mm, such as to slidingly receive a shaft with a major diameter of approximately 5 mm or less. Passageway 401 can comprise a hole or other passageway contained within tool holder 400. Passageway 401 can comprise a circular or non-circular cross section, such as a non-circular cross section constructed and arranged to prevent rotation of an inserted shaft portion 403.

Each tool holder 400 includes a set screw 402 (e.g. set screws 402a and 402b shown), which can be tightened to lock each tool holder 400 to main support frame 130. Set screws 402 can comprise a set screw with approximately 8-32 or 10-24 threads. In a loosened state of set screw 402, at least a portion of each associated tool holder 400 rotates about segment of main support frame 130 (e.g. rotates about an axis A_{SF} that is proximate the current position of tool holder 400 as shown). In some embodiments, tool holder 400 is constructed and arranged to rotate at least 360° about main support frame 130. In other embodiments, tool holder 400 is constructed and arranged to rotate less than 360°, such as a rotation less than 180° or less than 90°. Alternatively or additionally, in a loosened state of set screw 402, each associated tool holder 400 can slidingly translate along main support frame 130. In some embodiments, each set screw 402 can be further constructed and arranged to lock in place an elongate member (e.g. shaft portions 403a or 403b described herebelow) passing through the associated passageway 401.

In the embodiment of FIG. 4, a shaft portion 403a has been positioned within passageway 401a of tool holder 400a, and shaft portion 403b has been positioned within passageway 401b of tool holder 400b. Shaft portion 403a can comprise a tool guide (e.g. a hollow tube) used to support the shaft or other elongate portion of one or more tools, such as one or more of the surgical tools described hereabove. Alternatively, shaft portion 403a can comprise the shaft of a tool (e.g. one or more of the surgical tools described hereabove), wherein passageway 401a provides guiding and fixed orientation support to the inserted tool shaft. Shaft portion 403b is a shaft segment of cheek retractor 165, such that tool holder 400b allows positioning of cheek retractor 165. As described above, loosening of set screws 402a and 402b can allow repositioning relative to main support frame 130 (e.g. repositioning of the associated tool holder 400) of shaft portions 403a and 403b, respectively. Alternatively or additionally, loosening of set screws 402a and 402b can allow repositioning relative to the associated passageway 401 (e.g. sliding and/or rotating within the associated passageway 401) of shaft portion 403a and shaft portion 403b (i.e. cheek retractor 165), respectively. In some embodiments, shaft portion 403 is constructed and arranged to rotate at least 360° within passageway 401. In other embodiments, shaft portion 403 is constructed and arranged to rotate less than 360°, such as a rotation less than 180° or less than 90°.

In alternate embodiments, set screw 402 comprises a first set screw used to position and lock a tool holder 400 relative to main support frame 130, and a second set screw used to position and lock an elongate member within passageway 401, dual set screw configuration not shown but constructed and arranged to allow an operator to independently lock tool holder 400 and an elongate member positioned within passageway 401.

In some embodiments, passageway 401 comprises a first passageway constructed and arranged to slidingly receive a first elongate member and a second passageway constructed and arranged to receive a second elongate member, dual passageway configuration not shown but constructed and arranged to support two elongate members with similar or dissimilar cross sectional geometries. In these embodiments, separate set screws 402 can be used to fix the position of the associated elongate members within each passageway 401.

Tongue depressor blade 140 can include a tube 172 along at least a portion of its length (e.g. along at least a portion of the length of tongue depressor blade 140). In some embodiments, tube 172 is attachable to one or more tongue depressor blades 140, such as via one or more connecting elements, not shown but such as mating snaps or mating projections and grooves. Tube 172 can comprise a fluid attachment port, port 171, which can be constructed and arranged to operably attach to a vacuum and/or irrigation line. Tube 172 can comprise one or more openings, holes 173. Tube 172 and holes 173 can be constructed and arranged to provide and/or remove one or more fluids (e.g. liquids or gases) from and/or to an irrigation and/or vacuum source, respectively. In some embodiments, smoke (e.g. smoke created during delivery of energy to tissue) is removed through tube 172 via holes 173. In some embodiments, tube 172 comprises two or more lumens, such as two or more lumens which connect to similar and/or different holes 173.

Referring now to FIG. 5, a close-up cutaway side view of an articulation assembly of an oral retractor in a first position is illustrated, consistent with the present inventive concepts. Referring additionally to FIG. 5A, a close-up cutaway side view of the articulation assembly 200 of FIG. 5 in a second position is illustrated, consistent with the present inventive concepts. Referring additionally to FIG. 5B, a close-up view of a positioning assembly of FIGS. 5 and 5A, illustrating a rotational adjustment of tongue depressor blade 140, consistent with the present inventive concepts. Articulation assembly 200 can be constructed and arranged as part of oral retractor device 100 described hereabove with reference to FIG. 4. Therefore, a description of elements of oral retractor device 100 is not repeated for brevity. Articulation assembly 200 is positioned along the X, Y, and/or Z axes referred to in FIG. 4.

The articulation assembly 200 comprises a first portion 201 and a second portion 202 in communication with first portion 201. First portion 201 is also coupled to support arm 120 via an axle 205 extending along the Z axis.

Rotational positioning knob 125 comprises a gear 325 which rotatably engages a gear 301 coupled to a proximal end of first portion 201 (as shown in FIG. 5B). A rotation of positioning knob 125 drives an articulation of first portion 201 about axle 205 extending along the Z axis.

Second portion 202 of articulation assembly 200 is constructed and arranged to rotate relative to first portion 201 about an axle, axle 210, extending along the X axis (extending in and out of the sheet). Angular positioning knob 127 is constructed and arranged to act as a stop lock when second portion 202 rotates about axle 210, for example, shown at FIG. 5A. Second portion 202 comprises one or more arms 203 constructed and arranged to abut and slidingly receive tongue depressor blade 140. Tongue depressor blade 140 comprises a set of geared teeth 142 constructed and arranged to frictionally engage a gear, gear 326, of the multi-axis gear assembly coupled to an axle that extends along the X axis. Gear 326 can be coupled to height positioning knob 126 (shown in FIG. 5B), and can rotate about its axle in response to a rotation of height positioning knob 126. In doing so, the rotation of gear 326 engages geared teeth 142, which in turn moves tongue depressor blade 140 along a linear path thereby adjusting a vertical position of tongue depressor blade 140 relative to arms 203 of second portion 202 of articulation assembly 200.

Articulation assembly 200 comprises a locking assembly 250, including lever 251, axle 252, pin 253, and spring 254. Locking assembly 250 is constructed and arranged to, when in the locked position as shown in FIG. 5A, frictionally engage pin 253 with gear 326, locking the horizontal position of tongue depressor 140. In an unlocked position, as shown in FIG. 5, lever 251 is rotated (clockwise as shown) about axle 252, allowing spring 254 to pull pin 253 away from and disengaging gear 326. In the unlocked position, gear 326 is free to rotate such as to adjust the depth position of tongue depressor 140, for example, when inserted into the oral cavity of a patient for a medical procedure. In some embodiments, lever 251 can be constructed and arranged to be a quick release mechanism such as a mechanism released via a single finger or single hand of an operator.

In some embodiments, tongue depressor blade 140 is constructed and arranged to be removed while oral retractor device 100 is positioned in the patient's mouth. Tongue depressor blade 140 can be removed by sufficient rotation of height positioning knob 126 to cause tongue depressor blade 140 to translate vertically until geared teeth 142 disengage from gear 326. Alternatively or additionally, gear 326, geared teeth 142 and/or articulation assembly 200 can be constructed and arranged such that an operator can grip tongue depressor blade 140 (e.g. with a single hand), and apply a force sufficient to cause rotation of gear 326 and eventual disengagement of geared teeth 142 with gear 326, that is, with or without directly manipulating gear 326.

Referring now to FIGS. 6A-C, graphic demonstrations of a highly articulating probe device are illustrated, consistent with the present inventive concepts. A highly articulating robotic probe 550, according to the embodiment shown in FIGS. 6A-C, comprises essentially two concentric mechanisms, an outer mechanism and an inner mechanism, each of which can be viewed as a steerable mechanism. FIGS. 6A-C show the concept of how different embodiments of the articulating probe 550 operate. Referring to FIG. 6A, the inner mechanism can be referred to as a first mechanism or inner link mechanism 557. The outer mechanism can be referred to as a second mechanism or outer link mechanism 552. Each mechanism can alternate between being in rigid and limp states. In the rigid mode or state, the mechanism is just that—rigid. In the limp mode or state, the mechanism is highly flexible and thus either assumes the shape of its surroundings or can be re-shaped. It should be noted that the term “limp” as used herein does not necessarily denote a structure that passively assumes a particular configuration dependent upon gravity and the shape of its environment; rather, the “limp” structures described in this application are capable of assuming positions and configurations that are desired by the operator of the device, and therefore are articulated and controlled rather than flaccid and passive.

In some embodiments, one mechanism starts limp and the other starts rigid. For the sake of explanation, assume that in step 1 of FIG. 6A, outer link mechanism 552 is rigid and inner link mechanism 557 is limp. Now, inner link mechanism 557 is both pushed forward and its “head” or distal end is steered, as seen in step 2 in FIG. 6A. Now, inner link mechanism 557 is made rigid and outer link mechanism 552 is made limp. Outer link mechanism 552 is then pushed forward until it catches up or is coextensive with inner link mechanism 557, as seen in step 3 in FIG. 6A. Now, outer link mechanism 552 is made rigid, inner link mechanism 557 limp, and the procedure then repeats. One variation of this approach is to have outer link mechanism 552 be steerable as well. The operation of such a device is illustrated in FIG. 6B. In FIG. 6B it is seen that each mechanism is capable of catching up to the other and then advancing one link beyond. According to one embodiment, outer link mechanism 552 is steerable and inner link mechanism 557 is not. The operation of such a device is shown in FIG. 6C, illustrated in a series of steps.

In medical applications, once articulating probe 550 arrives at a desired location, the operator, typically a surgeon, can slide one or more tools through one or more working channels of outer link mechanism 552, inner link mechanism 557, or one or more working channels formed between outer link mechanism 552 and inner link mechanism 557, such as to perform various diagnostic and/or therapeutic procedures. In some embodiments, the channel is referred to as a working channel that can, for example, extend between first recesses formed in a system of outer links and second recesses formed in a system of inner links. Working channels may be included on the periphery of articulating probe 550, such as working channels comprising one or more radial projections extending from outer link mechanism 552, these projections including one or more holes sized to slidingly receive one or more tools.

In addition to medical procedures such as surgery, articulating probe 550 can be used in numerous applications including but not limited to: engine inspection, repair or retrofitting; tank inspection and repair; surveillance applications; bomb disarming; inspection or repair in tightly confined spaces such as submarine compartments or nuclear weapons; structural inspections such as building inspections; hazardous waste remediation; biological sample and toxin recovery; and combination of these. Clearly, the device of the present disclosure has a wide variety of applications and should not be taken as being limited to any particular application.

Inner link mechanism 557 and/or outer link mechanism 552 are steerable and inner link mechanism 557 and outer link mechanism 552 can each be made both rigid and limp, allowing articulating probe 550 to drive anywhere in three-dimensions while being self-supporting. Articulating probe 550 can “remember” each of its previous configurations and for this reason, articulating probe 550 can retract from and/or retrace to anywhere in a three dimensional volume such as the intracavity spaces in the body of a patient such as a human patient.

Inner link mechanism 557 and outer link mechanism 552 each include a series of links, i.e. inner links 556 and outer links 551 respectively, that articulate relative to each other. In some embodiments, outer links 551 are used to steer and lock the probe, while inner links 556 are used to lock the probe. In “follow the leader” fashion, while inner links 556 are locked, outer links 551 are advanced beyond a distal-most inner link. Outer links 551 are steered into position by the system steering cables, and then locked by locking the steering cables. The cable of inner links 556 is then released and inner links 556 are advanced to follow outer links 551. The procedure progresses in this manner until a desired position and orientation are achieved. The combined inner and outer links 551 include working channels for temporary or permanent insertion of tools at the surgery site. In some embodiments, the tools can advance with the links during positioning of the probe. In some embodiments, the tools can be inserted through the links following positioning of the probe.

One or more outer links 551 can be advanced beyond the distal-most inner link 556 prior to the initiation of an operator controlled steering maneuver, such that the quantity extending beyond the distal-most inner link 556 will collectively articulate based on steering commands. Multiple link steering can be used to reduce procedure time, such as when the specificity of single link steering is not required. In some embodiments, between 2 and 20 outer links 551 can be selected for simultaneous steering, such as between 2 and 10 outer links 551 or between 2 and 7 outer links 551. The number of links used to steer corresponds to achievable steering paths, with smaller numbers enabling more specificity of curvature of articulating probe 550. In some embodiments, an operator can select the number of links used for steering (e.g. to select between 1 and 10 links to be advanced prior to each steering maneuver).

Referring now to FIGS. 7A-D, side views of a series of shape adjustments to a robotically controlled tongue depressor blade including motor-driven hinges are illustrated, consistent with the present inventive concepts. Tongue depressor blade 140 of FIGS. 7A-D includes one or more hinges 175 (e.g. the three hinges 175a-c shown and positioned between segments of blade 140) which are operably attached to corresponding motors 176 (e.g. the three motors 176a-c shown), such as to rotate each hinge. Tongue depressor blade 140 can be of similar construction and arrangement as any tongue depressor blade 140 described herein. Each motor 176 can be connected (e.g. via one or more wires as shown) to a controller, such as controller 570 shown and described herein. Controller 570 can be operably connected to HID 575, also as shown and described herein, such that each segment of blade 140 positioned about a motor 176 can be rotated by an operator accessing HID 575, such that the overall curvature of blade 140 can be adjusted (e.g. the curvature provided by each segment of blade 140 is rotatable via one or more hinges 175). In some embodiments, motor 176 comprises a gear reduction assembly that provides precision of rotation of each hinge 175. Controller 570 and HID 575 can be part of an introducer assembly (e.g. an introducer assembly controlling an articulating probe), such as introducer assembly 500 described herein.

In some embodiments, the hinges 175 and motors 176 of tongue depressor blade 140 are constructed and arranged to allow an operator to transition blade 140 from a relatively straight geometry, to a continuously-curved geometry (such as is sequentially shown in FIGS. 7A-D), such as a continuously-curved geometry with an interior angle α₁ of less than or equal to 120°, such as an angle α₁ that is less than or equal to 110°, 100°, and/or 90°. Alternatively, hinges 175 can be operatively rotated such that blade 140 is in a discontinuous or other complex shape, such as the discontinuous shape shown in FIG. 7E. In some embodiments, controller 570 and HID 575 are constructed and arranged to adjust the position of tongue depressor blade 140 (e.g. with or without adjusting the shape of tongue depressor blade 140).

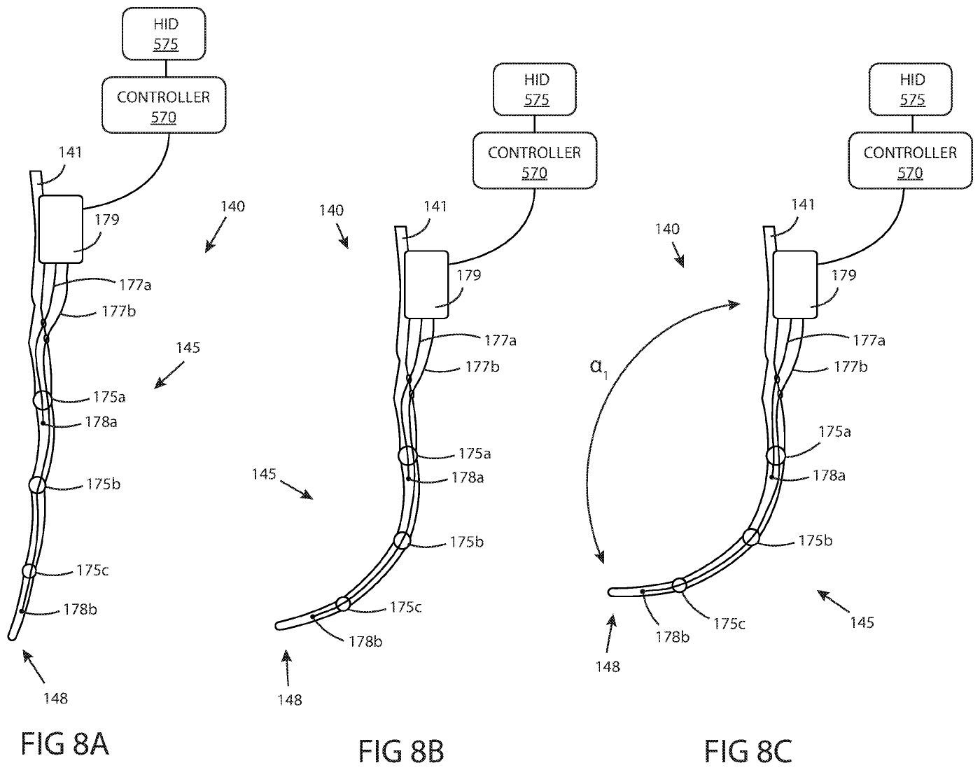

Referring now to FIGS. 8A-C, side views of a series of shape adjustments to a robotically controlled tongue depressor blade including cable driven segments are illustrated, consistent with the present inventive concepts. Tongue depressor blade 140 of FIGS. 8A-D includes one or more hinges 175 (e.g. the three hinges 175a-c shown and positioned between segments of blade 140). Tongue depressor blade 140 can be of similar construction and arrangement as any tongue depressor blade 140 described herein. One or more segments of blade 140 (as separated by each hinge 175) can be attached to a cable 177 (e.g. cables 177a and 177b as shown) at a weld, glue or other fixation points 178 (e.g. fixation points 178a and 178b, respectively, also as shown). Retraction of each cable 177 causes a segment of blade 140 to rotate about at least one hinge 175. In some embodiments, advancement of each cable 177 causes an opposite rotation of the associated segment of blade 140. Alternatively or additionally, one or more of these blade 140 segments can be biased (e.g. in a relatively linear orientation such as via a spring not shown), such that advancement or at least loosening of a cable 177 causes straightening of blade 140 (e.g. while retraction of a cable 177 causes blade 140 to curve). Each cable 177 can be operably connected on their proximal portion to a cable manipulation device, cable actuator 179 as shown (e.g. each cable 177 exiting blade 140 via one or more holes and subsequently entering cable actuator 179, as shown). Cable actuator 179 can comprise one or more motors and motor-driven bobbins (e.g. with or without a gear reduction assembly as described hereabove in reference to FIGS. 7A-D), such as to advance, retract, and/or control the tension of each cable 177. Cable actuator 179 can be operably connected (e.g. via one or more wires as shown) to a controller, such as controller 570 shown and described herein. Controller 570 can be operably connected to HID 575, also as shown and described herein, such that each segment of blade 140 attached to a cable 177 (e.g. via an anchor, fixation point 178) can be rotated by an operator accessing HID 575, such that the overall curvature of blade 140 can be adjusted (e.g. the curvature provided by each segment of blade 140 rotatable via one or more hinges 175). Controller 570 and HID 575 can be part of an introducer assembly (e.g. an introducer assembly controlling an articulating probe), such as introducer assembly 500 described herein.

In some embodiments, the hinges 175 and cables 177 of tongue depressor blade 140 are constructed and arranged to allow an operator to transition blade 140 from a relatively straight geometry, to a continuously-curved geometry (such as is sequentially shown in FIGS. 8A-D), such as a continuously-curved geometry with an interior angle α₁ of less than or equal to 120°, such as an angle α₁ that is less than or equal to 110°, 100°, and/or 90°. Alternatively, hinges 175 can be operatively rotated such that blade 140 is in a discontinuous shape, such as the discontinuous shape shown in FIG. 7E.

While the oral retractor device 100 and tongue depressor blade 140 have primarily been described for use in placement into the patient's mouth, placement in another natural orifice and/or into any incision into the patient should be considered within the spirit and scope of this application. Tongue depressor blade 140 can be positioned proximate (e.g. and apply force to) tissue of the patient's tongue, epiglottis, or any tissue internal to and/or external to the patient's body.

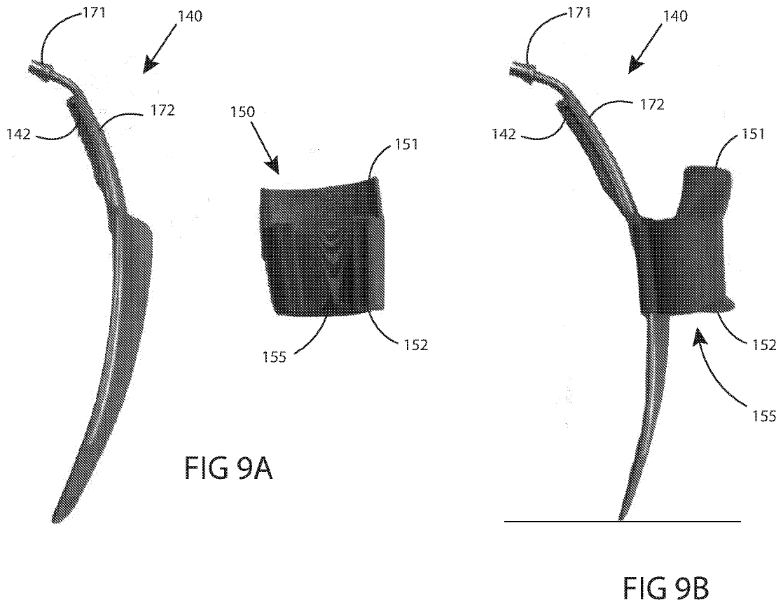

Referring now to FIGS. 9A and B, a side view of a tongue depressor blade and a tooth guard, and a side view of the tooth guard attached to the tongue depressor blade are illustrated, respectively, consistent with the present inventive concepts. Tongue depressor blade 140 can be constructed and arranged similar to that of tongue depressor blade 140 as described hereabove. System 10 can include a jaw manipulation element, tooth guard 150. Tooth guard 150 can comprise a proximal portion 151 and a distal portion 152, with a lumen 155 therethrough. In some embodiments, proximal portion 151 comprises a flared proximal end, as shown. Lumen 155 can be constructed and arranged to receive a surgical access device (e.g. an endoscope, a surgical robot, and/or other elongate access device) and/or a surgical instrument (e.g. graspers, cutters, and/or other elongate surgical tool). In some embodiments, tooth guard 150 is fixedly attached, or integral, to tongue depressor blade 140. In other embodiments, tooth guard 150 is removably attached to tongue depressor blade 140. In these embodiments, an operator (e.g. a surgeon or other medical professional) can adjust the position of tooth guard 150 along the length of tongue depressor blade 140 (e.g. adjust the position to accommodate patient anatomy).

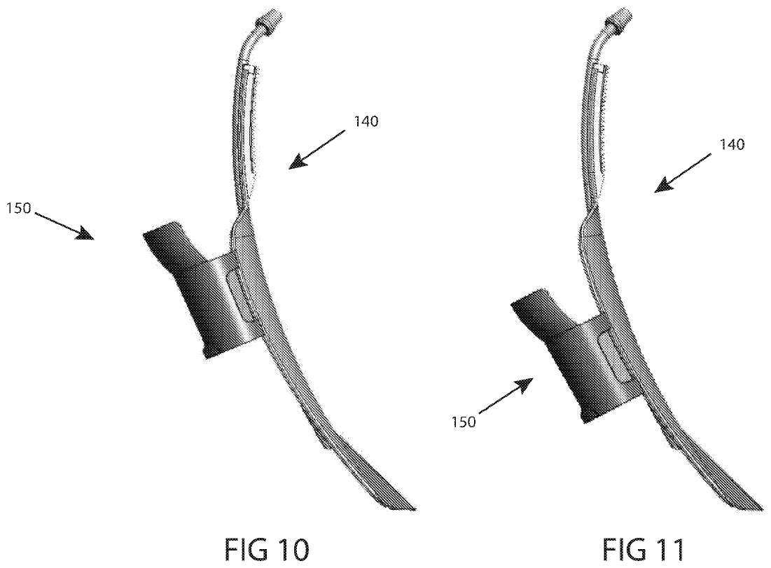

Referring now to FIG. 10, a side view of a tooth guard positioned on a proximal portion of a tongue depressor blade is illustrated, consistent with the present inventive concepts. Tooth guard 150 of FIG. 10 is positioned on a proximal portion of tongue depressor blade 140, as shown. In some embodiments, tooth guard 150 is fixedly attached to a proximal portion of tongue depressor blade 140. In other embodiments, tooth guard 150 is removably attached to a proximal portion of tongue depressor blade 140, such as is described above in reference to FIG. 9. In some embodiments, an operator (e.g. a surgeon or other medical professional) can adjust the position of tooth guard 150 along the length tongue depressor blade 140.

Referring now to FIG. 11, a side view of a tooth guard positioned on a distal portion of a tongue depressor blade is illustrated, consistent with the present inventive concepts. Tooth guard 150 of FIG. 11 is positioned on a distal portion of tongue depressor blade 140, as shown. In some embodiments, tooth guard 150 is fixedly attached to a distal portion of tongue depressor blade 140. In other embodiments, tooth guard 150 is removably attached to a distal portion of tongue depressor blade 140. In some embodiments, an operator (e.g. a surgeon or other medical professional) can adjust the position of tooth guard 150 along the length of tongue depressor blade 140.

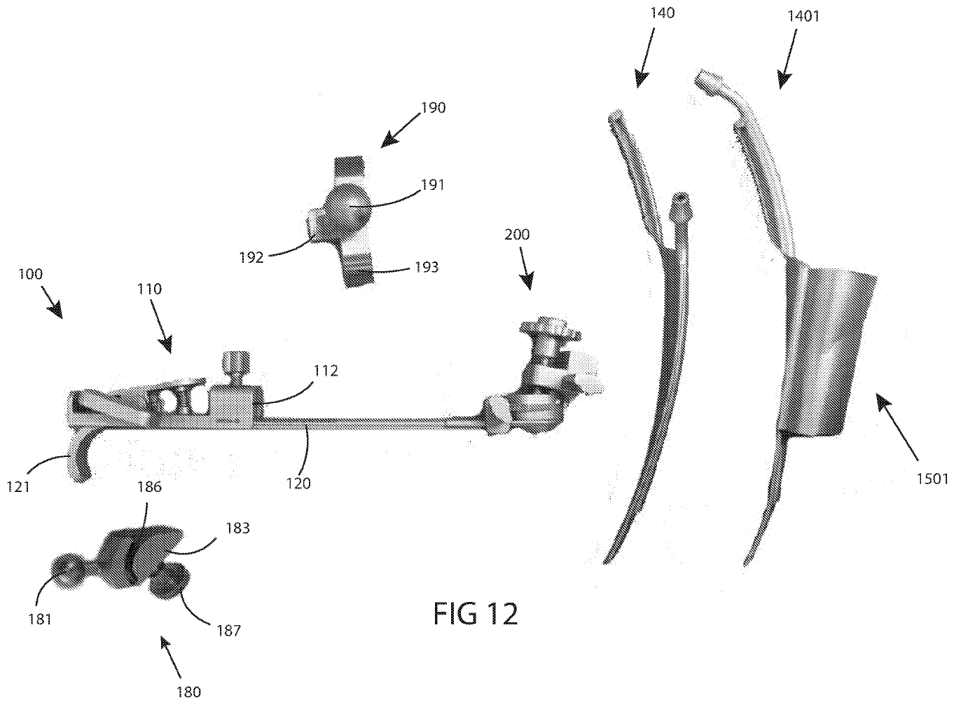

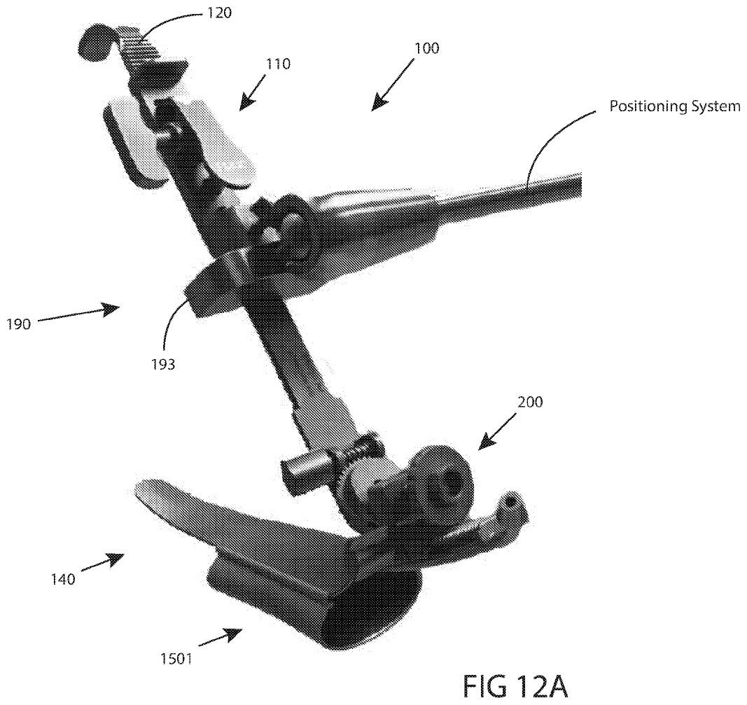

Referring now to FIG. 12, side views of a retraction device and tongue depressor blades, and a top view of an attachment mechanism are illustrated, consistent with the present inventive concepts. Referring additionally to FIG. 12A, a perspective view of a retraction device is illustrated, consistent with the present inventive concepts. Retraction device 100 can operably engage a tongue depressor blade, such as tongue depressor blade 140 or tongue depressor blade 1401 with an integrated tooth guard 1501, as shown. Retraction device 100 includes an articulation assembly 200 and linear positioning assembly 110 operably attached to a support arm 120. Articulation assembly 200 and linear positioning assembly 110 can each be constructed and arranged as described hereabove in reference to FIG. 1. Retraction device 100 can be removably attached to a positioning system (not shown, but such as a surgical positioning system attached to a surgical table) via an attachment mechanism 190. Attachment mechanism 190 can comprise a base 193 with a ball 191 and projection 192. Projection 192 can slidingly engage attachment point 112 of linear positioning assembly 110. The positioning system can slidingly receive ball 191 (as shown in FIG. 12A) and place the retraction device 100 into a fixed and stable position relative to the patient. Additionally or alternatively, retraction device 100 can include an adapter 180 for removably attaching support arm 120 to the positioning system. Adapter 180 can comprise a base 183, with recess 186 for slidingly receiving attachment portion 121 of support arm 120. Fastening device 187 can comprise a thumb screw, configured to secure adapter 180 to support arm 120. Adapter 180 can comprise a ball 191 for attaching to the positioning system. The fixed and stable position of retraction device 100 enables a gallows suspension of the patient to provide optical exposure of the endolarynx. Articulation assembly 200 can be constructed and arrange to articulate tongue depressor blade 140 while the patient is being placed in the suspended position and/or while in the suspended position, as described hereabove in reference to FIGS. 5, 5A, and 5B. Articulation assembly 200 can articulate tongue depressor blade 140 with at least three degrees of freedom. In some embodiments, linear positioning assembly 110 can be constructed and arranged to pull tongue depressor blade 140 towards attachment mechanism 190 (e.g. toward the support element, away from the surgical table), the function of linear positioning assembly 110 described hereabove in reference to FIG. 4. Alternatively, an operator can manually apply a force to pull tongue depressor blade 140 away from the surgical table, such as when adaptor 180 is attached to the positioning system, and linear positioning assembly 110 is not used in the positioning of tongue depressor blade 140.

Referring now to FIG. 13, a tongue depressor blade having a removable distal portion is illustrated, consistent with the present inventive concepts. Tongue depressor blade 1403 can comprise a distal end recess, recess 1402. Tongue depressor blade 1403 can include two or more removable and interchangeable distal portions, tips 1480, each comprising an engagement element, pins 1404. Recess 1402 can be configured to frictionally receive a pin 1404 and secure tip 1480 to tongue depressor blade 1403. In some embodiments, tongue depressor blade 1403 includes two or more interchangeable tips 1480, such as tips 1481-1485 shown. In some embodiments, tips 1481-1485 comprise a unique geometry (e.g. different lengths, widths, curvature, and/or overall shape). Tips 1481-1483 each comprise a triangular geometry. Tip 1484 comprises a projection with a spherical geometry, and tip 1485 comprises a projection with an ovoid geometry. In some embodiments, one or more of these geometries is provided in system 10 on an interchangeable tongue depressor blade 140, or as an interchangeable tip for a blade 1403. These geometries can be selected from by a clinician to best engage (e.g. atraumatically engage) a portion of the patient anatomy (e.g. the vallecula and/or the epiglottis). Interchangeable tips 1480, such as tips 1481-1485, can comprise a hollow geometry.

Referring now to FIGS. 14A and B, a side view and an end view of a tongue depressor blade are illustrated, respectively, consistent with the present inventive concepts. Tooth guard 150 comprises a height D1 (e.g. a distance of tooth guard 150 from the surface of blade 140). D1 can comprise a height of at least 10 mm and/or a height of no more than 25 mm. In some embodiments, tooth guard 150 comprises a relatively circular cross-sectional profile, such as when D1 represents the diameter of the cross section. Tooth guard 150 can comprise a length D2. D2 can comprise a length of at least 30 mm and/or a length of no more than 80 mm. The distal end of tooth guard 150 can be positioned a distance D3 from the distal end of blade 140. D3 can comprise a distance of at least 50 mm and/or at most 100 mm. Distal tip 1480 can comprise a length D4. D4 can comprise a length of at least 10 mm and/or no more than 50 mm. Distal tip 1480 can comprise a distal profile with a width D5 and a height D6. D5 can comprise a width between 5 mm and 16 mm, and D6 can comprise a height of no more than 15 mm.

Referring now to FIGS. 15A-C, side, top, and end views of a tongue depressor blade are illustrated, respectively, consistent with the present inventive concepts. Tooth guard 150 can comprise a height D1 (e.g. a distance of tooth guard 150 from the surface of blade 140). D1 can comprise a height of at least 10 mm and/or a height of no more than 50 mm, such as a height of 29.8 mm. In some embodiments, tooth guard 150 comprises a relatively circular and/or oblong cross-sectional profile. Tooth guard 150 can comprise a length D2 (e.g. a distance from the proximal end to the distal end of tooth guard 150). D2 can comprise a length of at least 30 mm and/or a length of no more than 65 mm, such as a length of 43.1 mm. The distal end of tooth guard 150 can be positioned a distance D3 from the distal end of blade 140. D3 can comprise a distance of at least 50 mm and/or a distance no more than 130 mm, such as a distance of 113.5 mm. Tooth guard 150 can comprise a proximal end width D9 of at least 25 mm and/or no more than 50 mm, such as a proximal end width of 37.6 mm. Tooth guard 150 can further comprise a distal end width D10 of at least 25 mm and/or no more than 50 mm, such as a distal end width of 38.9 mm. In some embodiments, tooth guard 150 comprises a taper from its proximal end to its distal end (e.g. the proximal end is larger than the distal end. In some embodiments, tooth guard 150 comprises an oblong shape, and lumen 155 extending through tooth guard 150 can comprise a height D12, less than width D11. Lumen 155 of tooth guard 150 can comprise a width D11 of at least 25 mm and/or no more than 50 mm, such as a width of 34.5 mm. Lumen 155 can further comprise a height D12 of at least 12 mm and/or no more than 40 mm, such as a height of 25.8 mm.

Blade 140 can comprise an overall length D7 (e.g. a distance from the proximal end of teeth 142 to the distal end of tip 1480). D7 can comprise a length of at least 125 mm and/or a length of no more than 255 mm, such as a length of 193.1 mm. Blade 140 and tooth guard 150 can comprise a collective depth D8 (e.g. the distance from the “top” of tip 1480 to the “bottom” of tooth guard 150). D8 can comprise a depth of at least 30 mm and/or a depth of no more than 65 mm, such as a depth of 48.9 mm.

Blade 140 can comprise a radius of curvature as described hereabove in reference to FIGS. 2, 2A-C, and 7A-D. In some embodiments, blade 140 comprises a continuous curve, such that blade 140 comprises a singular curve with a fixed radius. In other embodiments, blade 140 comprises a discontinuous or other complex curve, such as blade 140 comprises a relatively flat proximal portion and a curved distal end as described herein (as shown in FIG. 15A).

Blade 140 can include tube 172 with holes 173 to provide and/or remove one or more fluids (e.g. liquids or gases), as described hereabove in reference to FIG. 4. Blade 140 can include tube 172 with holes 173 to provide and/or remove one or more fluids (e.g. liquids or gases), as described hereabove in reference to FIG. 4. Blade 140 can comprise a light delivery element (e.g. a light emitting diode) and/or a camera, such as to provide illumination and/or to provide visualization of neighboring anatomy to an operator.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A retraction device for maintaining an opening into a patient's mouth and comprising:
a tissue manipulation element,
wherein the tissue manipulation element comprises a proximal portion with a first axis and a distal portion with a second axis,
wherein the first axis and the second axis define an interior angle, and
wherein the interior angle comprises an angle less than 120°.

2.-84. (canceled)
